(19) United States
(12) Patent Application Publication
(10) Pub. No.: US 2014/0121235 A1
(43) Pub. Date: May 1, 2014

(54) Method for Treating S6K-Overexpressing Cancers

(71) Applicant: Rush University Medical Center, Chicago, IL (US)

(72) Inventor: Xiulong Xu, Chicago, IL (US)

(73) Assignee: Rush University Medical Center, Chicago, IL (US)

(21) Appl. No.: 14/063,877

(22) Filed: Oct. 25, 2013

Related U.S. Application Data
(60) Provisional application No. 61/718,810, filed on Oct. 26, 2012.

(51) Int. Cl. A61K 31/42; A61K 31/437

(52) U.S. Cl. CPC A61K 31/42; A61K 31/437
USPC 514/300; 514/378

(57) ABSTRACT

Disclosed herein are methods for treating an S6K-overexpressing cancer, comprising administering a therapeutically effective amount of leflunomide to a subject in need thereof.

### RELATED APPLICATIONS

This application claims the benefit of the filing date under 35 U.S.C. §119(e) of Provisional U.S. Patent Application Ser. No. 61/718,810, filed Oct. 26, 2012, which is incorporated by reference in its entirety.

### TECHNICAL FIELD

The present invention relates to a method for treating S6K-overexpressing cancers.

### BACKGROUND

The phosphoinositide 3-kinase (PI3K) pathway is frequently activated in human cancers and plays essential roles in cell proliferation, apoptosis, protein synthesis, and metabolism. The PI3K pathway is activated through amplification or mutations of the genes encoding protein kinases or deletion of the tumor suppressor phosphatase and tensin homolog (PTEN) (1). In recent years, extensive efforts in developing the inhibitors of the PI3K pathway as novel therapeutic agents to treat certain types of cancer in which the PI3K pathway is hyperactivated have been thwarted by unacceptable toxicity or poor pharmacokinetics (2; 3). So far, only everolimus and temsirolimus, two rapamycin analogs that inhibit the mammalian target of rapamycin (mTOR), have been shown to be beneficial in several cancer types (2; 3).

Leflunomide (Arava™) is an immunomodulatory drug approved for the treatment of rheumatoid arthritis. Early studies revealed that the active metabolite of leflunomide, A77 1726, has two biochemical activities, the inhibition of tyrosine phosphorylation and inhibition of pyrimidine nucleotide synthesis (4-11). A77 1726 also inhibits the activation of the PI-3 kinase pathway in renal tubular cells infected with BK virus, leading to the inhibition of virus replication (16). However, the molecular target of A77 1726 in the PI-3 kinase pathway remains elusive; whether inhibition of the PI3K, pathway by A77 1726 contributes to its anti-proliferative activity is not known. In the present study, we report that A77 1726 is able to inhibit S6K1 activation in several solid tumor cell lines, subsequently inducing the feedback activation of AKT phosphorylation and the MAP kinase pathway largely through the IGF-1 receptor.

### SUMMARY

Provided herein is a method for treating an S6K-overexpressing cancer, comprising administering a therapeutically effective amount of leflunomide to a subject in need thereof. Leflunomide may inhibit or reduce cell proliferation. Leflunomide may inhibit or reduce activity of an S6K protein. The S6K protein may be S6K1. Leflunomide may inhibit or reduce phosphorylation of a ribosomal S6 protein. Leflunomide may induce phosphorylation of a protein selected from the group consisting of AKT, S6K1, ERK1/2, and MEK.

The S6K-overexpressing cancer may be a breast cancer, endometrial cancer, pancreatic neuroendocrine tumor, bladder cancer, renal cell carcinoma, myeloma. The S6K-overexpressing cancer may have an amplification of an S6K gene or a hyperactivated mTOR-S6K1 pathway.

The method may further comprise administering a therapeutically effective amount of an agent. The agent in combination with leflunomide may reduce the proliferation of cancer cells more than leflunomide alone. The agent may be PLX4720.

### BRIEF DESCRIPTION OF THE DRAWINGS

FIG. 1 shows the effect of A77 1726 on the PI-3 and MAP kinase pathways is independent of its anti-pyrimidine synthesis activity. A375 cells seeded in a 6-well plate were starved in DMEM medium containing 0.5% FBS for 2 hr and treated with the indicated concentration of A77 1726 or PLX 4720 (1 μM) for another 2 hr (A) or treated with A77 1726 (100 μM) for the indicated time (B & C). Cells were harvested and analyzed for protein phosphorylation by specific antibodies as indicated. Protein loading was monitored by stripping membrane and re-probing with antibodies against non-phosphorylated proteins. (D) Uridine fails to reverse the effect of A77 1726 on the PI-3 and MAP kinase pathways. A375 cells seeded in a 6-well plate were starved in DMEM medium containing 05% FBS for 2 hr and treated with the indicated concentration of A77 1726 in the absence or presence of uridine (200 μM). Phosphorylated and total proteins were analyzed by Western blot as described above.

FIG. 2 shows the ability of leflunomide and A77 1726 to inhibit S6K1 in vitro. Leflunomide and A77 1726 diluted at the final concentrations as indicated were pre-mixed with S6K1 for 30 min, followed by the addition of peptide substrate of S6K1 and incubation for 1 hr. S6K1 activity was measured by using a ADP-Glo™ system. The experiment was repeated with similar results. The data represents the mean±standard deviation from one experiment in triplicate.

FIG. 3 shows the feedback activation of the PI-3 and MAP kinase pathway through IGF-1 receptor. A375 cells seeded in 6-well plates were starved in DMEM medium containing 0.5% FBS for 2 hr and then pre-treated with vehicle (0.1% dimethyl sulfoxide), LY294002 (10 μM) (A), PPP (1 μM) (B), or the inhibitors of the MAP kinase pathway (PLX4720, 1 μM; U0126, 10 μM; PD98059, 10 μM) (C) for 1 hr. Cells were then treated with A77 1726 (100 μM) or rapamycin (20 nM) as indicated for 2 hr. Cells were harvested and analyzed for protein phosphorylation by specific antibodies as indicated. Protein loading was monitored by stripping membrane and re-probing with antibodies against non-phosphorylated proteins.

FIG. 4 shows the anti-proliferative effect of A77 1726. A375 cells were seeded in 96-well plates (2000 cells/well) and incubated the indicated concentrations of A77 1726 for 72 hr in the absence or presence of various concentrations PLX4720 (A, C) with or without uridine (200 μM) (B, D). Cell proliferation was analyzed by an ATP-based Cell-Glo assay. One representative of three experiments with similar results was shown. *p<0.01.

FIG. 5 shows the effect of exogenous uridine and MAP kinase pathway inhibitors on A77 1726-stimulated S phase entry and cell cycle arrest. (A) Ability of uridine to relieve the cell cycle arrest in the S phase. A375 cells grown in 6-well plates were treated with the indicated concentration of A771726 with or without uridine (200 μM) for 24 hr. Cell cycle was analyzed in a flow cytometer as described in Materials and Methods. (B) Effect of the MAP kinase pathway inhibitors on A77 1726-mediated cell cycle arrest in the S phase. A375 cells were treated with A771726 (100 μM) in the absence or presence of 0.1% DMSO, PIA 4720 (1 μM), U0126 (10 μM), PD98059 (10 for 24 hr. Single cell suspensions were prepared and analyzed for cell cycle in a flow cytometer. (C) Effect of the PI-3 kinase pathway inhibitors and their effect on A77 1726-mediated cell cycle arrest. A375 cells were treated with A77 1726 (100 μM), rapamycin (20 nM), and/or LY294002 (10 μM). After incubation for 24 hr, cells were harvested and analyzed for cell cycle progress.

FIG. 6 shows the effect of exogenous uridine and MAP kinase pathway inhibitors on A77 1726-stimulated BrdU incorporation. (A) The effect of uridine to A77 1726-induced DNA synthesis. A375 cells were treated with the indicated concentration of A771726 in the absence or presence of uridine (200 μM) for 22 hr. After pulsing with BrdU (10 UM) for 2 hr, cells were harvested and analyzed for BrdU incorporation by staining with an Alexa Fluor-488-conjugated anti-BrdU monoclonal antibody followed by flow cytometry. (B) Effect of the MAP kinase pathway inhibitors to A77 1726-stimulated BrdU incorporation. A375 cells were treated with A77 1726 (100 μM) in the presence of 0.1% DMSO, PLX4720 (1 μM), U0126 (10 UM), PD98059 (10 μM). After incubation for 22 hr the cells were pulsed with BrdU for 2 hr. Single cell suspensions were stained for BrdU incorporation and analyzed for cell cycle in a flow cytometer as described in (A). (C) Effect of the PI-3 kinase pathway inhibitors and their effect on A77 1726-stimualted BrdU incorpration. A375 cells were treated with A771726 (100 μM), rapamycin (20 nM), and/or LY249002 (10 μM). After incubation for 22 hr, cells were pulsed with BrdU for 2 hr, harvested and analyzed for BrdU labeling in a flow cytometer.

FIG. 7 shows the feedback activation of MAP and PI-3 kinase pathway in A375 and MCF-7 cells but not in BT-20 cells. (A) A77 1726 induces MAP kinase activation in A375 cells cultured in 10% fetal bovine serum. A375 cells were seeded in a 6-well plate overnight and then stimulated with various concentrations of A77 1726 or PLX 4720 (1 μM) for 2 hr. Cells were harvested and analyzed for MAP kinase activation by anti-phospho-ERK1/2 antibody. The membrane was stripped and re-probed with anti-ERK1/2 antibody. (B) Lack of the feedback activation of the MAP and PI-3 kinase pathways in BT-20 cells. BT20 cells seeded in 6-well plates were starved in DMEM medium containing 05% EBS for 2 hr and treated with A77 1726 (0, 50 or 100 μM), Cells were harvested and analyzed for protein phosphorylation by specific antibodies as indicated. Protein loading was monitored by stripping membrane and re-probing with antibodies against non-phosphorylated proteins. (C) Strong feedback activation of the PI-3 kinase pathway and weak feedback activation of the MAP kinase pathway in MCF-7 cells by A77 1726 and rapamycin. MCF-7 cells seeded in a 6-well plate were starved in the medium containing 0.5% fetal bovine serum for 3 hr and then left untreated or treated with A77 1726 (100 μM) or rapamycin (20 nM) for 2 hr. Cells were harvested and analyzed for protein phosphorylation with the indicated antibodies. (D) The feedback activation of the PI-3 and MAP kinase pathway by leflunomide effect of leflunomide. A375 cells seeded in 6-well plates were starved in DMEM medium containing 05% FBS for 3 hr and treated with leflunomide (0, 5, 10, or 100 μM) for 2 hr. Cells were harvested and analyzed for protein phosphorylation by specific antibodies as indicated. Protein loading was monitored by stripping membrane and re-probing with antibodies against nonphosphorylated proteins.

FIG. 8 shows the feedback activation of PI-3 and MAP kinase pathway by leflunomide. A375 cells seeded in 6-well plates were starved in DMEM medium containing 05% EBS for 2 hr and then pre-treated with LY294002 (10 μM) or PPP (1 μM) for 1 hr. Cells were then stimulated with leflunomide for 2 hr. Cells were harvested and analyzed for protein phosphorylation by specific antibodies as indicated. Protein loading was monitored by stripping membrane and re-probing with antibodies against non-phosphorylated proteins.

FIG. 9 shows the time-dependent effect of A77 1726 on cell cycle progress. A375 cells grown in 6-well plates were treated with A77 1726 (50 μM) for the indicated lengths of time. Single cell suspensions were prepared and fixed with 2 ml cold 70% ethanol in PBS overnight at 4° C. Cells were then treated with RNase A followed by labeling with propidium iodine. Cell cycle was analyzed in a flow cytometer.

FIG. 10 shows the differential effects of A77 1726 on cell cycle progress in MCF-7 and BT-20. MCF-7 and BT-20 grown in 6-well plates were treated with the indicated concentration of A771726 for 24 hr (A). Single cell suspensions were prepared and fixed with 2 ml cold 70% ethanol in PBS overnight at 4° C. Cells were then treated with RNase A followed by labeling with propidium iodine. Cell cycle was analyzed in a flow cytometer.

FIG. 11 shows the effect of leflunomide on cell cycle progress. A375 cells grown in 6-well plates were treated with the indicated concentration of leflunomide for 24 hr. Single cell suspensions were prepared and fixed with 2 ml cold 70% ethanol in PBS overnight at 4° C. Cells were then treated with RNase A followed by labeling with propidium iodine. Cell cycle was analyzed in a flow cytometer.

FIG. 12 illustrates inhibition of carbamoyl-phosphate synthetase 2 (CAD) by A 77 1726.

FIG. 13 illustrates inhibition of cyclin 1) expression by A 77 1726.

### DETAILED DESCRIPTION

The present invention relates to methods for treating an S6K-overexpressing cancer. An S6K-overexpressing cancer may include cancers that have increased activity of an S6K protein. Such cancers may include breast cancer, endometrial cancer, pancreatic neuroendocrine tumor, bladder cancer, renal cell carcinoma, and myeloma. Increased activity of the S6K, protein may result from increased levels of S6K protein, increased levels of an S6K mRNA transcript, amplification of an S6K gene, and/or altered levels of phosphorylation of the S6K protein.

The methods of the present invention may include administering to a subject suffering from an S6K-overexpressing cancer a therapeutically effective amount of leflunomide. The inventors have discovered that leflunomide may inhibit or reduce cell proliferation by inhibiting or reducing the activity of an S6K protein. Particularly, the S6K protein may be S6K1, also known as p70 S6. In addition to inhibiting S6K activity, leflunomide may inhibit or reduce phosphorylation of ribosomal S6 protein, but may induce phosphorylation of AKT, S6K1, ERK1/2, and MEK. In other words, inhibition of S6K activity may lead to reduced phosphorylation of ribosomal S6 protein, and to feedback activation of the MAP kinase pathway and upstream segments of the PI-3 kinase pathway, thereby exerting an anti-proliferative effect on S6K-overexpressing cancerous cells.

The methods of the present invention may also include administering to the subject suffering from an S6K-overexpressing cancer a therapeutically effective amount of an agent. The agent in combination with leflunomide may reduce proliferation of S6K-overexpressing cancerous cells more than leflunomide alone. The agent may be rapalogs, metformin, thalidomide, B-raf tyrosine kinase inhibitors such as PLX4720. Alternatively, the agent may be any molecule (e.g., small molecule, peptide, antibody, nucleic acid, etc.) that exerts an anti-proliferative effect on cancerous cells either alone and/or in combination with leflunomide.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. METHODS FOR TREATING S6K-OVEREXPRESSING CANCERS

Provided herein is a method for treating an S6K-overexpressing cancer. The method may include administering to a subject suffering from an S6K-overexpressing cancer a therapeutically effective amount of lefunomide. Leflunomide may modulate (e.g., inhibit or reduce) S6K activity to inhibit or reduce the proliferation of cancer cells overexpressing S6K.

a. S6K-overexpressing Cancers

The S6K-overexpressing cancer may include cancers that have increased activity of an S6K protein. Increased activity of the S6K protein may result from increased levels of S6K protein, increased levels of an S6K mRNA transcript, amplification of an S6K gene (i.e., change in S6K, gene copy number), and/or altered levels of phosphorylation of the S6K protein. The S6K protein may be a substrate of mammalian target of rapamycin (mTOR), a serine/threonine kinase. Together S6K and mTOR may regulate multiple aspects cell physiology, for instance, cell growth, proliferation, and metabolism. S6K may phosphorylate ribosomal S6 protein. Additionally, in human cells, S6K may be known as p70 S6 kinase or S6K1.

Such S6K-overexpressing cancers may include breast cancer. S6K1 gene amplification occurs in 10% of breast cancers (22). Cancers not overexpressing S6K may include, but are not limited to, certain types of ovarian cancer, endometrial cancer, pancreatic neuroendocrine tumor, bladder cancer, renal cell carcinoma, myeloma, certain types of prostate cancer certain types of glioblastoma, and/or certain types of non small cell lung cancer.

b. Lefinnomide

The methods for treating an S6K-overexpressing cancer may include administering to a subject suffering from such a cancer a therapeutically effective amount of lefunomide. Leflunomide may also be known as SU101 or ARAVA. An active metabolite of leflunomide may include A77 1726 or SU0020.

Leflunomide, including its metabolites A77 1726, may inhibit or reduce cell proliferation by inhibiting or reducing the activity of an S6K protein. Particularly, the S6K protein may be S6K1, also known as p70 S6. In addition to inhibiting S6K activity, leflunomide may inhibit or reduce phosphorylation of ribosomal S6 protein, but may induce phosphorylation of AKT, S6K1, ERK1/2, and MEK. In other words, inhibition of S6K activity may lead to reduced phosphorylation of ribosomal S6 protein, and to feedback activation of the MAP kinase pathway and upstream segments of the PI-3 kinase pathway, thereby exerting an antiproliferative effect on S6K-overexpressing cancerous cells.

(1) Other Agents in Combination with Leflunomide

The methods of the present invention may also include administering to the subject suffering from an S6K-overexpressing cancer a therapeutically effective amount of an agent. The agent in combination with leflunomide may reduce proliferation of S6K-overexpressing cancerous cells more than leflunomide alone. In other words, inhibition of proliferation of S6K-overexpressing cancerous cells by leflunomide and the agent may be additive. The agent may be PLX4720. Alternatively, the agent may be any molecule (e.g., small molecule, peptide, antibody, nucleic acid, etc.) that exerts an anti-proliferative effect on cancerous cells either alone and/or in combination with leflunomide. The agent may be any molecules that indirectly leads to inhibition of S6K activity, such as rapamycin, rapalogs, metformin, resveratrol,

c. Pharmaceutical Compositions

Leflunomide may be incorporated into pharmaceutical compositions suitable for administration to a subject (such as a patient, which may be a human or non-human). The pharmaceutical compositions may include a “therapeutically effective amount” or a “prophylactically effective amount” of leflunomide. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of the composition are outweighed by the therapeutically beneficial effects. A “prophylactically effective amount” refers to an amount effective, at dosages and tar periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of lefitunomide may include a loading dose and a maintenance dose. The loading dose may be administered prior to administration of the maintenance dose. The loading dose may be about 1 mg/day to about 2000 mg/day, about 2 mg/day to about 1900 mg/day, about 3 mg/day to about 1800 mg/day, about 4 mg/day to about 1700 mg/day, about 5 mg/day to about 1600 mg/day, about 10 mg/day to about 1500 mg/day, about 15 mg/day to about 1400 mg/day, about 20 mg/day to about 1300 mg/day, about 25 mg/day to about 1200 mg/day, about 30 mg/day to about 1100 mg/day, about 35 mg/day to about 1000 mg/day, about 40 mg/day to about 900 mg/day, about 45 mg/day to about 800 mg/day, about 50 mg/day to about 700 mg/day, about 55 mg/day to about 600 mg/day, about 60 mg/day to about 500 mg/day, about 65 mg/day to about 450 mg/day, about 70 mg/day to about 425 mg/day, about 75 mg/day to about 400 mg/day, about 80 mg/day to about 375 mg/day, about 85 mg/day to about 350 mg/day, about 90 mg/day to about 325 mg/day, about 95 mg/day to about 300 mg/day, about 96 mg/day to about 275 mg/day, about 97 mg/day to about 250 mg/day, about 98 mg/day to about 225 mg/day, about 99 mg/day to about 200 mg/day, about 100 mg/day to about 175 mg/day, and about 125 mg/day to about 150 mg/day. The loading dose may be administered for about 1 day, about 2 days, about 3 days, about 4 days, about 6 days, and about 7 days. Particularly, the loading dose may be about 100 mg/day for 3 days. Alternatively, no loading dose may be administered.

The maintenance dose may be about 0.1 mg/day to about 500 mg/day, about 0.5 mg/day to about 450 mg/day, about 1 mg/day to about 400 mg/day, about 2 mg/day to about 350 mg/day, about 3 mg/day to about 300 mg/day, about 4 mg/day to about 250 mg/day, about 5 mg/day to about 200 mg/day, about 6 mg/day to about 150 mg/day, about 7 mg/day to about 100 mg/day, about 8 mg/day to about 95 mg/day, about 9 mg/day to about 90 mg/day, about 10 mg/day to about 85 mg/day, about 11 mg/day to about 80 mg/day, about 12 mg/day to about 75 mg/day, about 13 mg/day to about 70 mg/day, about 14 mg/day to about 65 mg/day, about 15 mg/day to about 60 mg/day, about 16 mg/day to about 55 mg/day, about 17 mg/day to about 50 mg/day, about 18 mg/day to about 45 mg/day, about 19 mg/day to about 40 mg/day, about 20 mg/day to about 35 mg/day, and about 25 mg/day to about 30 mg/day.

The maintenance dose may be administered for a time period of about 1 week to about 10 years, about 2 weeks to about 9.5 years, about 3 weeks to about 9 years, about 4 weeks to about 8.5 years, about 5 weeks to about 8 years, about 6 weeks to about 7.5 years, about 7 weeks to about 7 years, about 8 weeks to about 6.5 years, about 9 weeks to about 6 years, about 10 weeks to about 5.5 years, about 11 weeks to about 5 years, about 12 weeks to about 4.5 years, about 13 weeks to about 4 years, about 14 weeks to about 3.5 years, about 15 weeks to about 3 years, about 16 weeks to about 2.5 years, about 17 weeks to about 2 years, about 18 weeks to about 1.5 years, and about 19 weeks to about 1 year. The maintenance dose may be administered for multiples of such time periods in which no leflunomide is administered between the multiple time periods. Alternatively, the maintenance dose may be administered until S6K-overexpressing cancer cells are not present in the subject or not detectable in the subject. Alternatively, no maintenance dose may be administered after administration of the loading dose.

In another example, the loading dose may be about 1 mg/m²/day to about 2000 mg/m²/day, about 2 mg/m²/day to about 1900 mg/m²/day, about 3 mg/m²/day to about 1800 mg/m²/day, about 4 mg/m/day to about 1700 mg/m²/day, about 5 mg/m²/day to about 1600 mg/m²/day, about 10 mg/m²/day to about 1500 mg/m²/day, about 15 mg/m²/day to about 1400 mg/m²/day, about 20 mg/m²/day to about 1300 mg/m²/day, about 25 mg/m²/day to about 1200 mg/m²/day, about 30 mg/m²/day to about 1100 mg/m²/day, about 35 mg/m²/day to about 1000 mg/m²/day, about 40 mg/m²/day to about 900 mg/m²/day, about 45 mg/m²/day to about 800 mg/m²/day, about 50 mg/m²/day to about 700 mg/m²/day, about 55 mg/m²/day to about 600 mg/m²/day, about 60 mg/m²/day to about 500 mg/m²/day, about 65 mg/m²/day to about 450 mg/m²/day, about 70 mg/m²/day to about 425 mg/m²/day, about 75 mg/m²/day to about 400 mg/m²/day, about 80 mg/m²/day to about 375 mg/m²/day, about 85 mg/m²/day to about 350 mg/m²/day, about 90 mg/m²/day to about 325 mg/m²/day, about 95 mg/m²/day to about 300 mg/m²/day, about 96 mg/m²/day to about 275 mg/m²/day, about 97 mg/m²/day to about 250 mg/m²/day, about 98 mg/m²/day to about 225 mg/m²/day, about 99 mg/m²/day to about 200 mg/m²/day, about 100 mg/m²/day to about 175 mg/m²/day, and about 125 mg/m²/day to about 150 mg/m²/day. The loading dose may be administered for about 1 day, about 2 days, about 3 days, about 4 days, about 6 days, and about 7 days. Particularly, the loading dose may be about 200 mg/m²/day for about 4 days or about 400 mg/m²/day for about 4 days. Alternatively, no loading dose may be administered.

In a further example, the maintenance dose may be about 1 mg/m²/day to about 2000 mg/m²/day, about 2 mg/m²/day to about 1900 mg/m²/day, about 3 mg/m²/day to about 1800 mg/m²/day, about 4 mg/m²/day to about 1700 mg/m²/day, about 5 mg/m²/day to about 1600 mg/m²/day, about 10 mg/m²/day to about 1500 mg/m²/day, about 15 mg/m²/day to about 1400 mg/m²/day, about 20 mg/m²/day to about 1300 mg/m²/day, about 25 mg/m²/day to about 1200 mg/mL/day, about 30 mg/m²/day to about 1100 mg/m²/day, about 35 mg/m²/day to about 1000 mg/m²/day, about 40 mg/m²/day to about 900 mg/m²/day, about 45 mg/mL/day to about 800 mg/m²/day, about 50 mg/m²/day to about 700 mg/m²/day, about 55 mg/m²/day to about 600 mg/m²/day, about 60 mg/m²/day to about 500 mg/m²/day, about 65 mg/m²1 day to about 450 mg/m²/day, about 70 mg/m²/day to about 425 mg/m²/day, about 75 mg/m²/day to about 400 mg/m²/day, about 80 mg/m²/day to about 375 mg/m²/day, about 85 mg/m²/day to about 350 mg/m²/day, about 90 mg/m²/day to about 325 mg/m²/day, about 95 mg/m²/day to about 300 mg/m²/day, about 96 mg/m²/day to about 275 mg/m²/day, about 97 mg/m²/day to about 250 mg/m²/day, about 98 mg/m²/day to about 225 mg/m²/day, about 99 mg/m²/day to about 200 mg/m²/day, about 100 mg/m²/day to about 175 mg/mL/day, and about 125 mg/m²/day to about 150 mg/m²/day.

The maintenance dose may be administered for a time period of about 1 week to about 10 years, about 2 weeks to about 9.5 years, about 3 weeks to about 9 years, about 4 weeks to about 8.5 years, about 5 weeks to about 8 years, about 6 weeks to about 7.5 years, about 7 weeks to about 7 years, about 8 weeks to about 6.5 years, about 9 weeks to about 6 years, about 10 weeks to about 5.5 years, about 11 weeks to about 5 years, about 12 weeks to about 4.5 years, about 13 weeks to about 4 years, about 14 weeks to about 3.5 years, about 15 weeks to about 3 years, about 16 weeks to about 2.5 years, about 17 weeks to about 2 years, about 18 weeks to about 1.5 years, and about 19 weeks to about 1 year. The maintenance dose may be administered for multiples of such time periods in which no leflunornide is administered between the multiple time periods. Particularly, the maintenance does may be about 200 mg/m²/day for about 10 weeks or about 400 mg/m²/day for about 10 weeks. Alternatively, the maintenance dose may be administered until S6K-overexpressing cancer cells are not present in the subject or not detectable in the subject. Alternatively, no maintenance dose may be administered after administration of the loading dose.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

d. Modes of Administration

Methods for treating S6K-overexpressing cancers may include any number of modes of administering leflunomide or pharmaceutical compositions of leflunomide. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixiers, solid emulsions, solid dispersions or dispersible powders. For the preparation of pharmaceutical compositions for oral administration, leflunomide may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, leflunomide may also be dispersed in a microparticle, e.g. a nanoparticulate, composition.

For parenteral administration, leflunomide or pharmaceutical compositions of leflunomide can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration leflunomide or pharmaceutical compositions of leflunomide can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

The present invention has multiple aspects, illustrated by the following non-limiting examples.

### 3. EXAMPLES

### Example I

### Materials and Methods for Examples 2-4

Reagents.

Leflunomide and A77 1726 were kindly provided by Cinkate Corporation (Oak Park, Ill.). PPP were purchased from Santa Cruz Biotechnology, Inc. (Santa Cruz, Calif.). Bay 43-9006 was kindly provided by Bayer Pharmaceuticals, Inc. (Pittsburg, Pa.). PD98059, U0126, LY294002, rapamycin, and the following antibodies were purchased from Cell Signaling Technology (Danvers, Mass.), including ERK1/2, MEK1/2, Raf-1, p90 RSK, GSKα/β, 4E-BP, PDK1, AKT, AKT, mTOR, S6K1, S6. Their corresponding phosphor-antibodies include ERK1/2^{T202/Y204}, MEK1/2^{S217/S221}, Raf-1^{S338}, p90 RSK^{T356/S360}, GSKα/β^{S21/9}, 4E-BP^{T37/46}, PDK^{S241}, AKT^{S473}, AKT^{T308}, mTOR^{S2448}S6K1^{T389}, S6^{S235/236}.

Cell Lines.

A375 is a melanoma cell line with BRAE^{V600E} mutation, wild-type PTEN/PI3KC and p53. MCF-7 is an estrogen-positive breast cancer cell line with PI3KC mutation but with wild-type p53. MDA-MB-231 is a breast cancer cell line with both KRAS (K13D) and BRAF (G464V) gene mutations, and p53 (R280K) mutation. BT-20 is a breast cancer cell line with PI3KC mutations (P539R and H1047R) and p53 (K132Q) mutation. A375 and BT-20 cells were grown in the complete DMEM medium supplemented with 10% fetal bovine serum, streptomycin and penicillin, L-glutamine. MCF-7 cells were grown in the complete MEM medium supplemented with 10% fetal bovine serum, streptomycin and penicillin, L-glutamine, non-essential amino acids, Hepes buffer, and. All four cell lines were purchased from American Tissue Culture Collection (Manassas, Va.).

Western Blot.

Cells grown in 6-well plates were harvested and lysed in NP-40 lysis buffer (50 mM Tris-HCI (pH 8.0), 150 mM NaCl, 1% NP-40, 5 mM EDTA, 10 μg/ml aprotinin, 10 μg/ml leupeptin, and 1 mM phenylmethylsulfonyl fluoride). After incubation on ice for 30 min, the cell lysates were prepared by spinning down at 4° C., 15,000 rpm for 15 min. After electrophoresis and transfer to Immobolin membrane, proteins of interest were probed with their specific antibodies, followed by horseradish peroxidase-conjugated goat anti-rabbit IgG and SuperSignal Western Pico enhanced chemiluminoscence substrate (Pierce Chemical Co., Rockford, Ill.).

In Vitro p70 S6 Kinase Assay.

The ability of lefiunomide and A77 1726 to inhibit p70 S6 kinase assay was conducted by using an ADP-Glo™ Kinase assay system (Promega Corporation, Madison, Wis.). Briefly, A77 1726 or leflunomide diluted in the kinase buffer was mixed with recombinant p70 S6 kinase (100 ng/reaction) and incubated at room temperature for 30 min. Peptide substrate of p70 S6 kinase (5 μg/reaction) and ATP (10 μM, final concentration) was added, with a total final volume of 25 μl. After incubation for 1 hr, ADP-Glo™ reagent (25 μl) was added to each reaction. After incubation for 40 min, kinase detection substrate (luciferin) (50 μl/reaction) was added. After incubation fix 30 min, luciferase activity was measured by reading in a luminescence plate reader. The experiment was conducted in triplicate and repeated once with similar results. The data from one experiment were presented as mean±standard deviation.

Cell Proliferation Assay.

A375 cells were grown in complete RPMI 1640 medium supplemented with 10% fetal bovine serum. The cells were seeded in 96-well plates at the density of 2,000/well in the absence or presence of various concentrations of A77 1726, PLX4720, or uridine (200 μM). After incubation for 72 hr, cell proliferation was monitored by using an ATP-based Cell-Glo assay (Promegan, Madison, Wis.) following the manufacturer's instruction.

DNA Replication and Cell Cycle Analysis.

Cells were grown in complete RPMI1640 medium with 10% fetal bovine serum in 6-well plates. Upon 60% confluence, the cells were treated with vehicle or with indicated concentrations of A77 1726 or various inhibitors as indicated for 24 hr. Cells were pulsed with 10 μM BrdU for 2 hr. Cells were harvested, denatured with 2N HCl for 5 min at room temperature followed by neutralization with 0.1M borate buffer (pH 8.5). After washing and blocking with normal mouse serum, the cells were stained with an Alexa Fluor 488-conjugated anti-BrdU monoclonal antibody (BD Bioscience), followed by analysis in a flow cytometer. Alexa Fluor 488-conjugated mouse IgG was included as a control. For cell cycle analysis, the cells were harvested and fixed with 2 ml cold 70% ethanol in PBS overnight at 4° C. Fixed cells were then washed 3 times with PBS, treated with RNaseA (100 μg/ml in 0.5 ml PBS). After incubation at room temperature for 30 min, cells were stained with 2.5 μl propidium iodine (10 mg/ml.) and immediately analyzed for DNA content in a flow cytometer.

Statistical Analyses.

Unpaired student t test was used to determine if there was a significant difference in the cell proliferation. A p value <0.05 was considered statistically significant. All statistics were performed with SigmaPlot 11 software (Richmond, Calif.).

### Example 2

### Effect of A77 1726 on the PI-3 and MAP Kinase Pathways

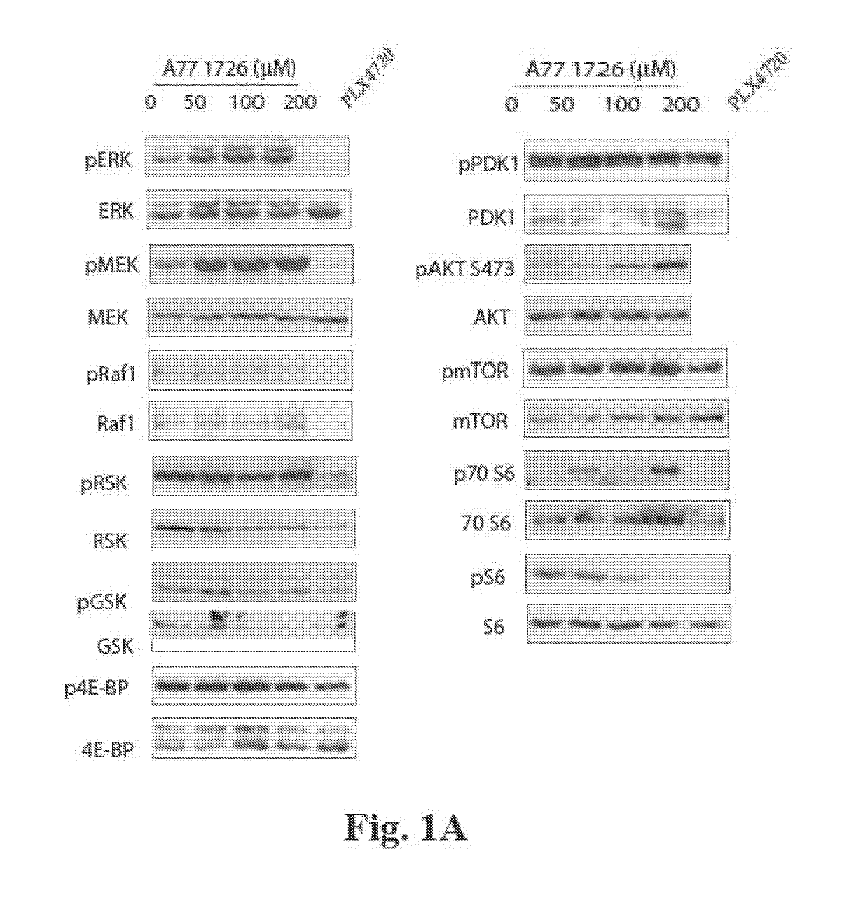

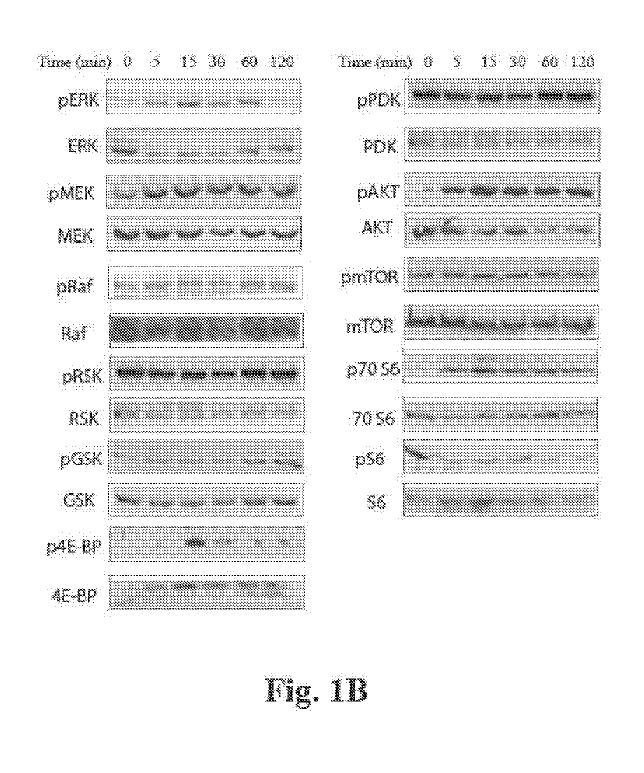

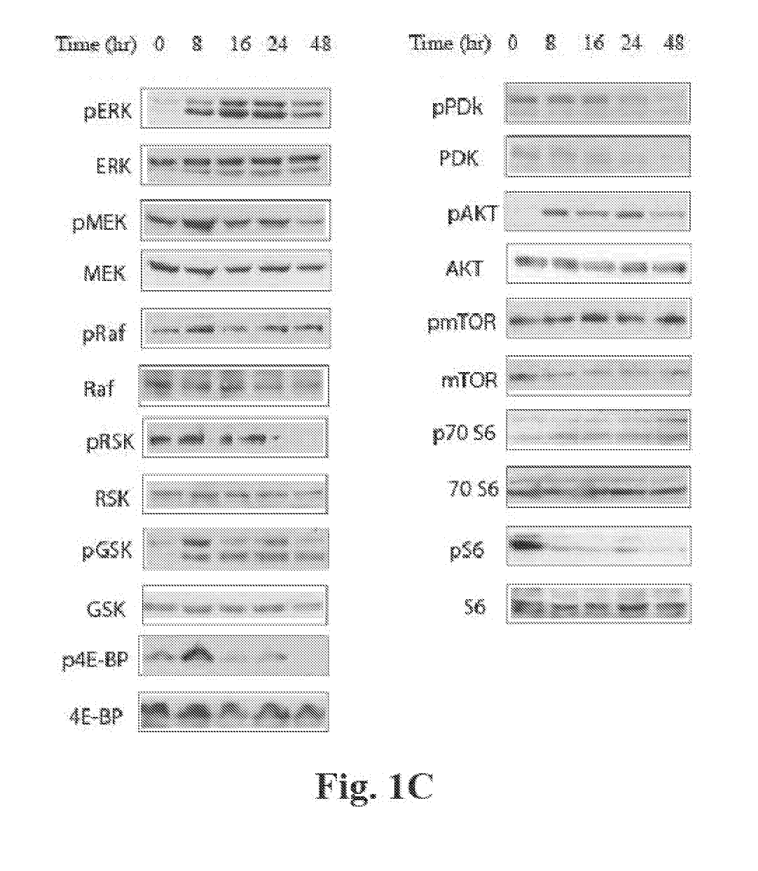

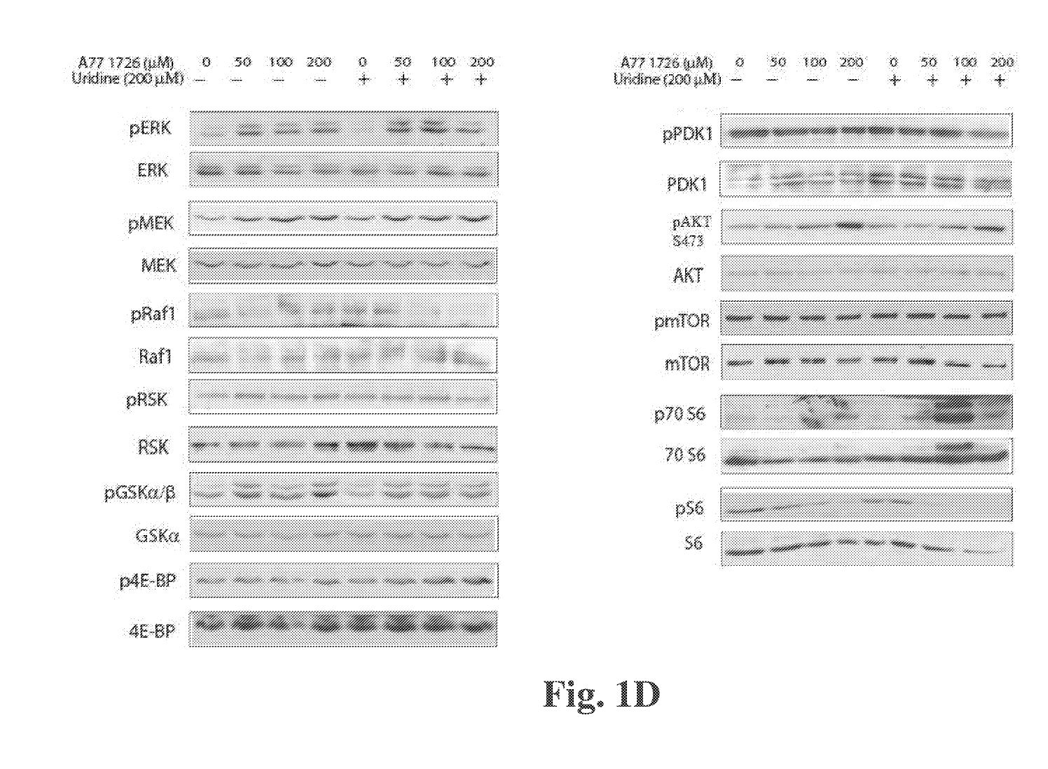

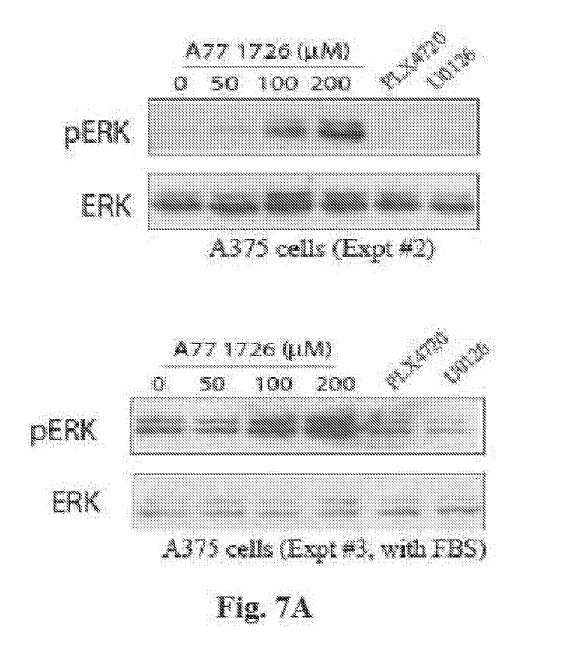

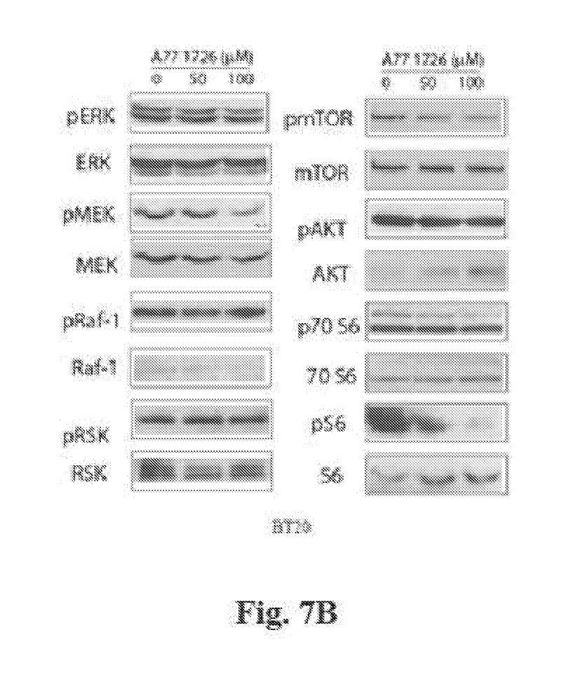

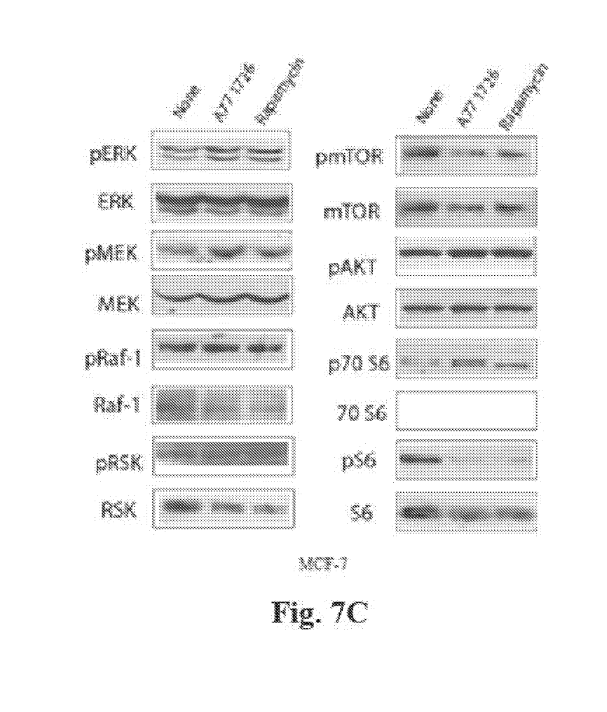

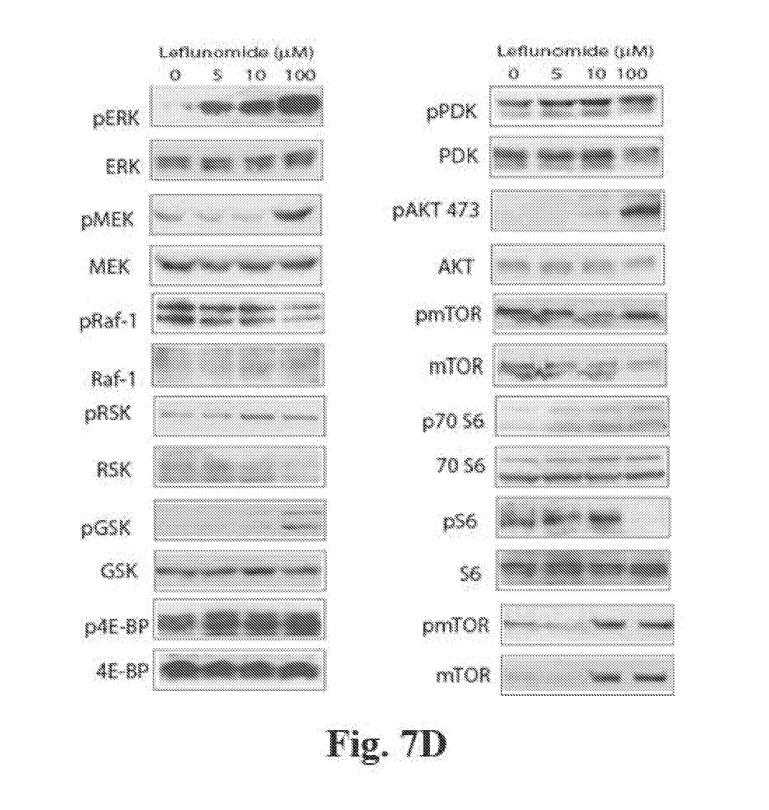

The PI-3 and MAP kinase pathways play an important role in cell proliferation, differentiation, and cell cycle progress. Multiple genes in these two pathways are frequently mutated and have a commanding role in driving tumorigenesis and tumor cell proliferation (2; 3). Previous tudies demonstrated that A77 1726 was able to inhibit growth factor receptor tyrosine kinase activities (5-7; 18). Here we tested whether A77 1726 affected the activation of MAP and PI-3 kinase pathways. A375 cells grown in 6-well plates were starved in the media containing 0.5% FBS for 2 hr and then treated with the indicated concentration of A77 1726. A77 1726 strongly induced ERK1/2^{T202/204} and MEK^{S217/S221} phosphorylation but had no effect on Raf-1 phosphorylation in A375 cells (FIG. 1A). In contrast, the B-Raf kinase inhibitor PLX 4720 (1 μM) inhibited ERK1/2 and MEK phosphorylation in A375 cells (FIG. 1A). Induction of ERK1/2 phosphorylation was similarly achieved in A77 1726-treated A375 cells cultured in the medium containing 10% fetal bovine serum (FIG. 7A). A77 1726 slightly induced ERK phosphorylation in MCF-7 cells (FIG. 7B) but had no effect on BT-20 cells (FIG. 7C). We next explored the effect of A77 1726 in phosphorylation of the molecules in the PI-3 kinase pathway. A77 1726 strongly induced phosphorylation of AKT^{S473} and S6K1^{T389} in a dose-dependent manner and but had no or only weal or minimal effect on phosphorylation of PDK1^{S241}, MTOR^{S2448}, GSKα/β^{S21/9}, p90 RSK^{T353/356}, and 4E-BP^{T3/46}. In contrast, A77 1726 inhibited the phosphorylation of ribosomal protein S6^{S235/S236} in a dose-dependent manner (FIG. 1A). Inhibition of S6^{S235/236} phosphorylation was also observed in BT-20 and MCF-7 cells (FIGS. 7B and 7C). Similar results were obtained with leflunomide (FIG. 7D), except that it was slightly more potent than A77 1726 in inducing a feedback activation of the MAP kinase pathway. Induction of AKT^{S473} and S6K1^{T389} phosphorylation was very quick (FIG. 1B) and lasted up to 24 hr (FIG. 1C). Inhibition of S6^{S235/S236} phosphorylation was also long-lasting, started within few minutes after (FIG. 1B) exposure to A77 1726 and lasted for up to 48 hr (FIG. 1C).

A77 1726 is a potent inhibitor of pyrimidine nucleotide synthesis. To rule out the possibility that the activation of PI-3 and MAP kinase pathways was not due to pyrimidine nucleotide depletion, we tested whether the addition of exogenous uridine that can normalize pyrimidine nucleotide levels (5; 6) was able to affect phosphorylation in the MAP and PI-3 kinase pathways. As shown in FIG. 1D, uridine (200 μM) was unable to block A77 1726-induced phosphorylation of ERK1/2^{T202/Y204}, MEK1/2^{S217/S221} AKT^{S473}S6K1^{T389}, GSKα/β^{S21/9}, and P90RSK^{T353/356}, and unable to restore S6^{S235/236} phosphorylation. These observations suggest that the effect of A77 1726 on the MAP and PI-3 kinase pathways is not mediated through its inhibitory effect on pyrimidine nucleotide synthesis.

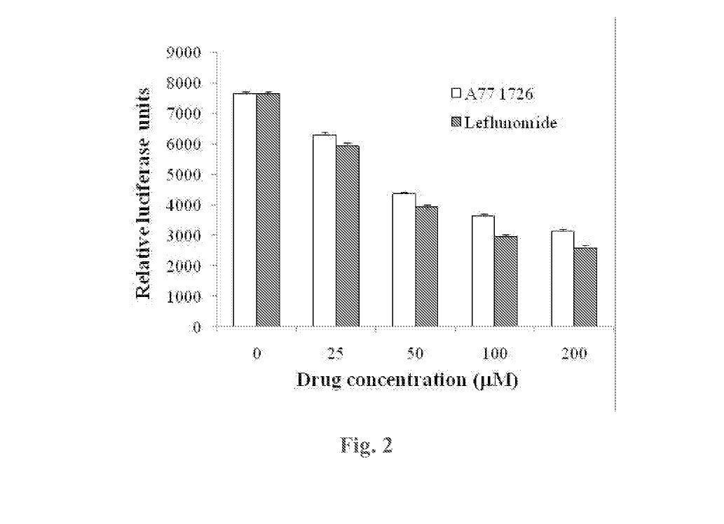

The ability of A77 1726 to inhibit S6 but to increase AKT and S6K1 phosphorylation strongly suggests that S6K1 is the molecular target of A77 1726. To prove this, we conducted an in vitro kinase assay to test whether leflunomide and A77 1276 were able to directly inhibit S6K1 activity. Indeed, leflunomide and A77 1726 both inhibited the activity of recombinant S6K1 in a dose-dependent manner, with the 10₅₀ values of approximately 55 and 80 μM, respectively (FIG. 2).

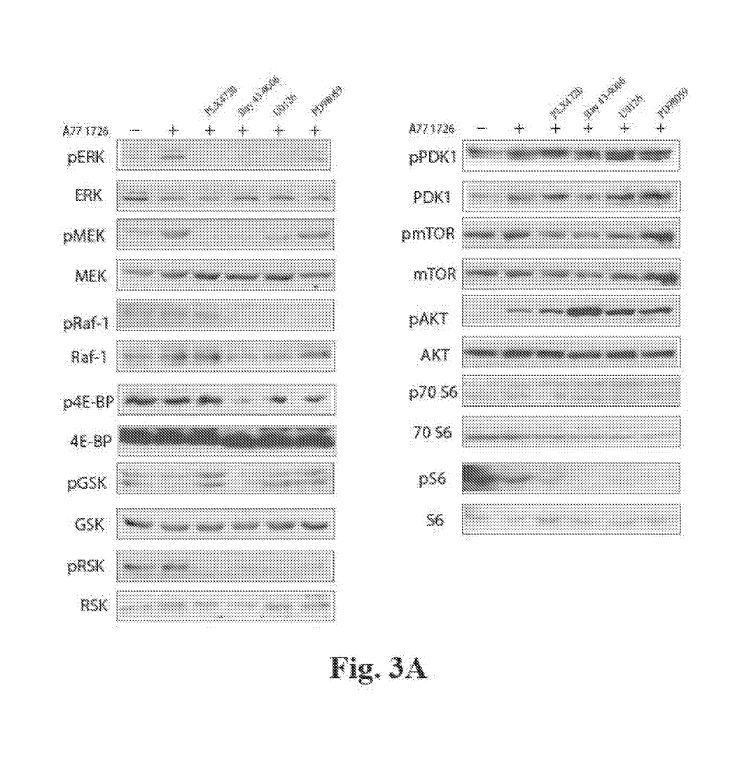

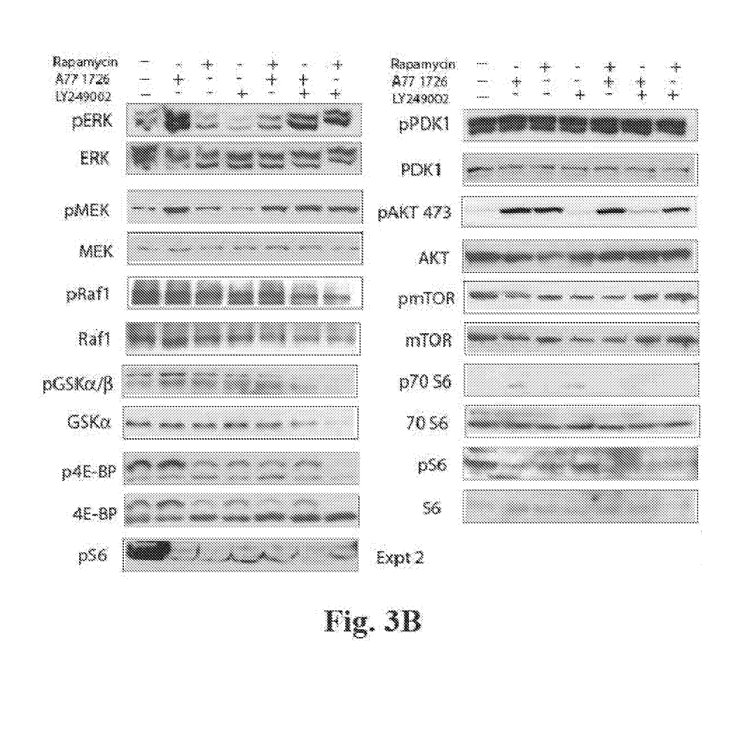

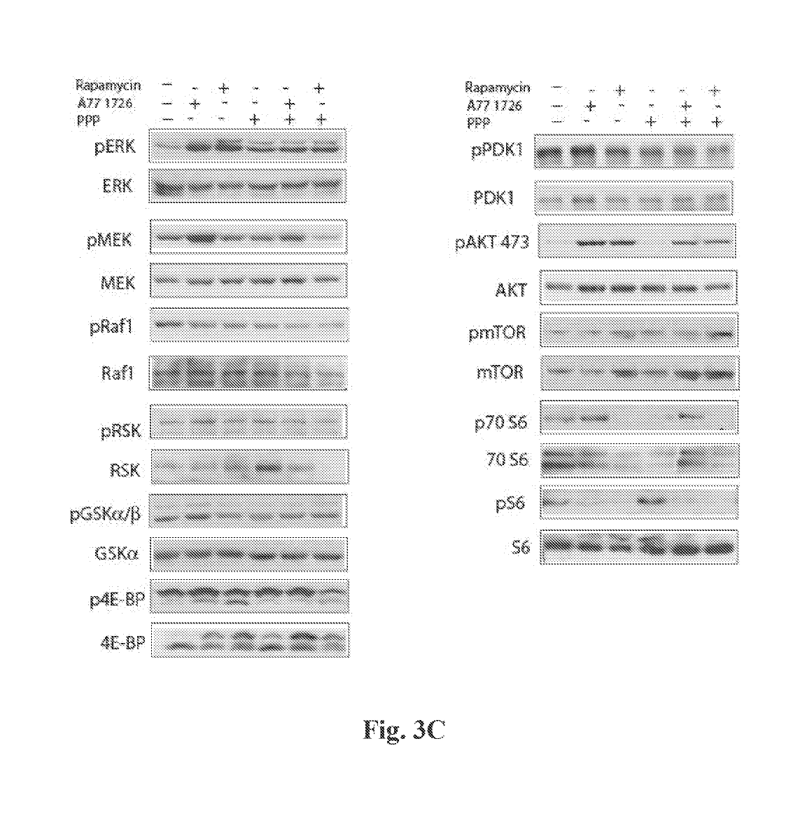

We next determined whether the inhibitors of the MAP kinase pathway were able to inhibit A77 1726-induced MAP kinase pathway activation in A375 cells. Two inhibitors of Ruf kinase (PLX4720 and BAY 43-9006) and a MEK inhibitor (U0126) were able to block A77 1726-induced phosphorylation of ERK1/2^{T202/Y204} and MEK1/2^{S217/S221}. PD98059, an inhibitor of ERK1/2, was unable to block A77 1726-induced ERK1/2^{T202/Y204} and MEK1/2^{S}217/S221 phosphorylation (FIG. 3A). Interestingly, inhibition of the MAP kinase pathway by PLX4720, Bay 43-9006, U0126, and PD98059 led to further increase of AKT^{S473} phosphorylation. Among them, BAY 4-9006 was the most effective inhibitor in potentiating AKT^{S473} phosphorylation. These inhibitors had weak effect on A77 1726-induced S6K1 phosphorylation and inhibited GSKα/β^{S21/9}, 4E-BP^{T37/46} and SO^{S235/236} phosphorylation (FIG. 3B), reflecting the complexity of multiple-layer cross-talk between the PI-3 and MAP kinase pathways.

The above observations suggest that the feedback activation of the MAP kinase pathway is mediated through an upstream molecule such as Ras or IGF-1 receptor. A prior study demonstrated that rapamycin-mediated feedback activation of the MAP kinase pathway is mediated through PI-3 kinase-induced Ras activation in MCF-7 cells (19). Here we tested whether PI-3 kinase was also involved in A77 1726-induced MAP kinase pathway activation. We found that LY294002, a PI-3 kinase inhibitor, had little effect on A77 1726- and rapamycin-induced phosphorylation of MEK and ERK1/2 (FIG. 3B). However, LY294002 was able to largely block A77 1726- and rapamycin-induced AKT^{S473} phosphorylation (FIG. 3B). LX294002 also potentiated A77 1726 and raparmycin-mediated inhibition of S6^{S235/236} phosphorylation. Of note, rapamycin inhibited S6K1 phosphorylation and blocked A77 1726-induced S6K1 phosphorylation (FIG. 3B). Similar results were observed in MCF-7 breast cancer cell line (data not shown). These results suggest that A77 1726-mediated feedback activation of the MAP kinase pathway is largely independent of PI-3 kinase and could be mediated through an upstream receptor tyrosine kinase.

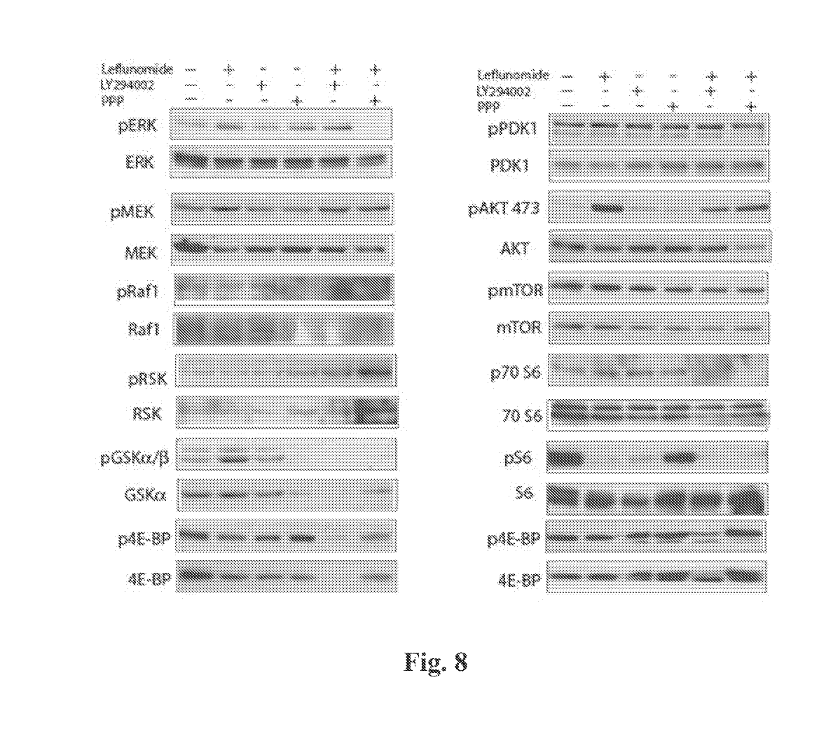

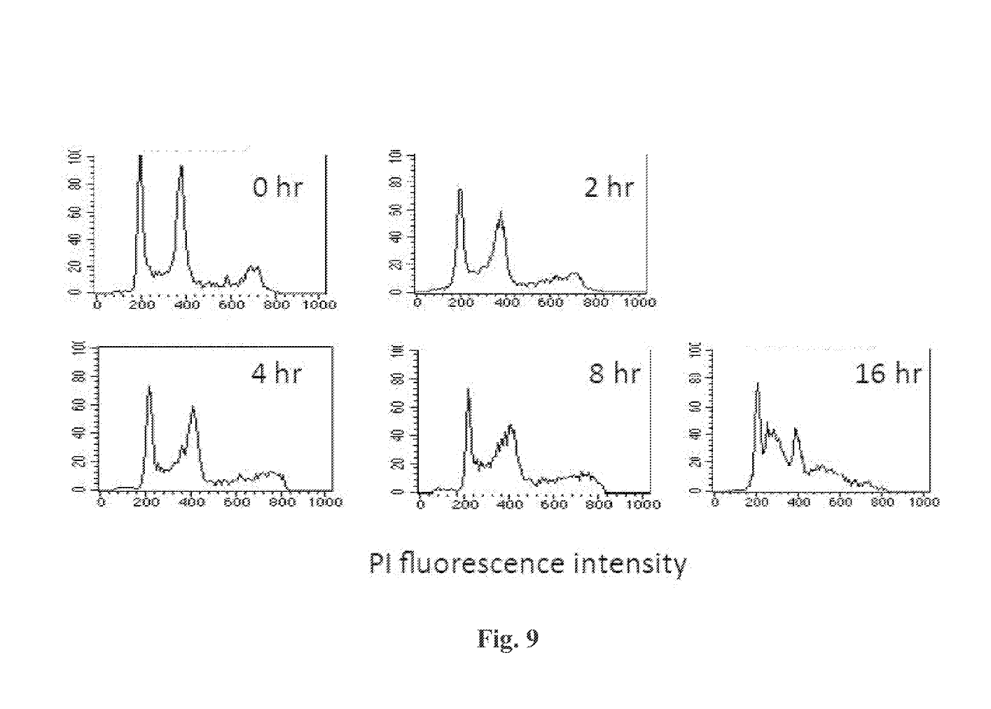

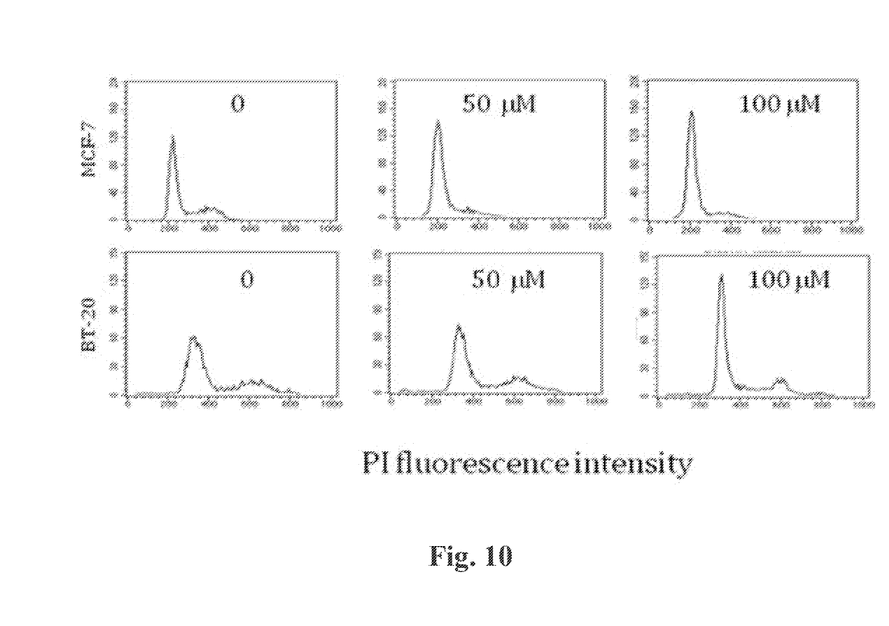

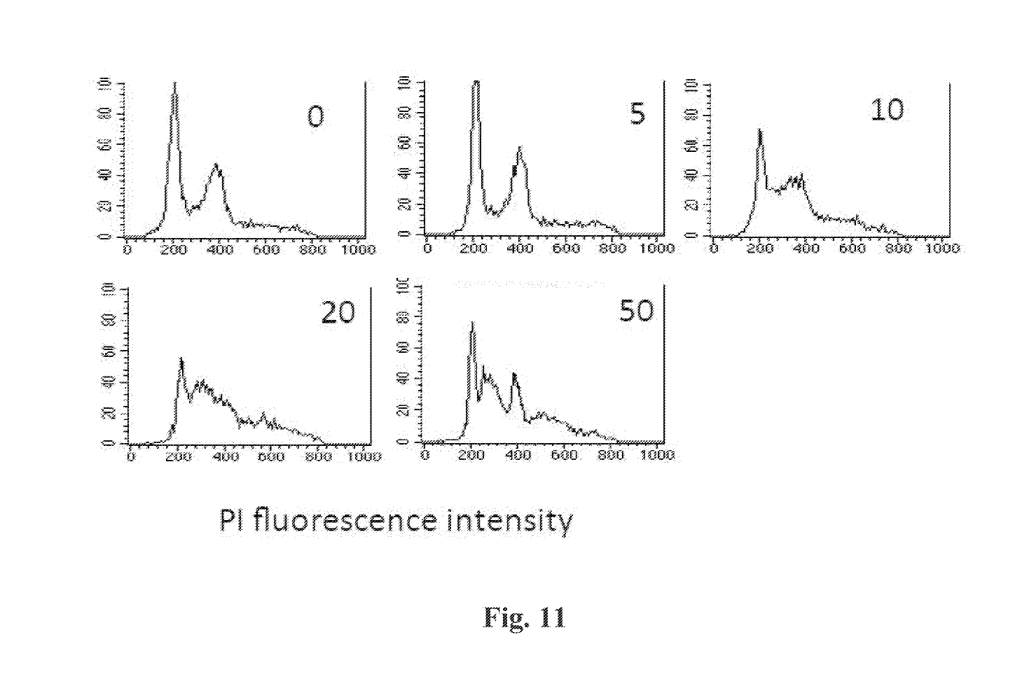

It is well established that insulin receptor substrate (IRS) in the IGF-1 receptor signaling pathway can be negatively regulated by S6K1 (2). Thus, we tested whether inhibition of IGF-1 receptor tyrosine kinase activity led to the suppression of A77 1726-induced PI-3 and MAP kinase pathway activation. As shown in FIG. 3C, A77 1726 induced. ERK1/2^{T202/Y2041} and MEK1/2^{S217/S221} phosphorylation. Both A77 1726 and rapamycin inhibited S6^{S235/S236} phosphorylation but induced AKT^{T473} phosphorylation. PPP, a specific inhibitor of IGF-1 receptor, alone had no effect on ERK1/2^{T202/Y204}, MED1/2^{S217/S221}, AKT^{T473}, and S6^{S235/236} phosphorylation but abrogated A77 1726-induced ERK1/2^{T202/Y204} and MEK1/2^{S217/S221} phosphorylation. PPP also largely blocked A77 1726- and rapamycin-induced AKT^{T473} phosphorylation. Weak induction of A77 1726-induced GSKα/β^{S21/9}, p90 RSK^{T353/356}, and 4E-BP^{T37/46} phosphorylation in A375 cells was also blocked by PPP. These results suggest that A77 1726-induced feedback activation of the PI-3 and MAP kinase pathways is largely mediated through the IGF-1 receptor. Similar observations were observed with PPP and LY294002 on leflunomide-induced feedback activation of the PI-3 and MAP kinase pathway (FIG. 8).

### Example 3

### Anti-Proliferative Effect of A77 1726

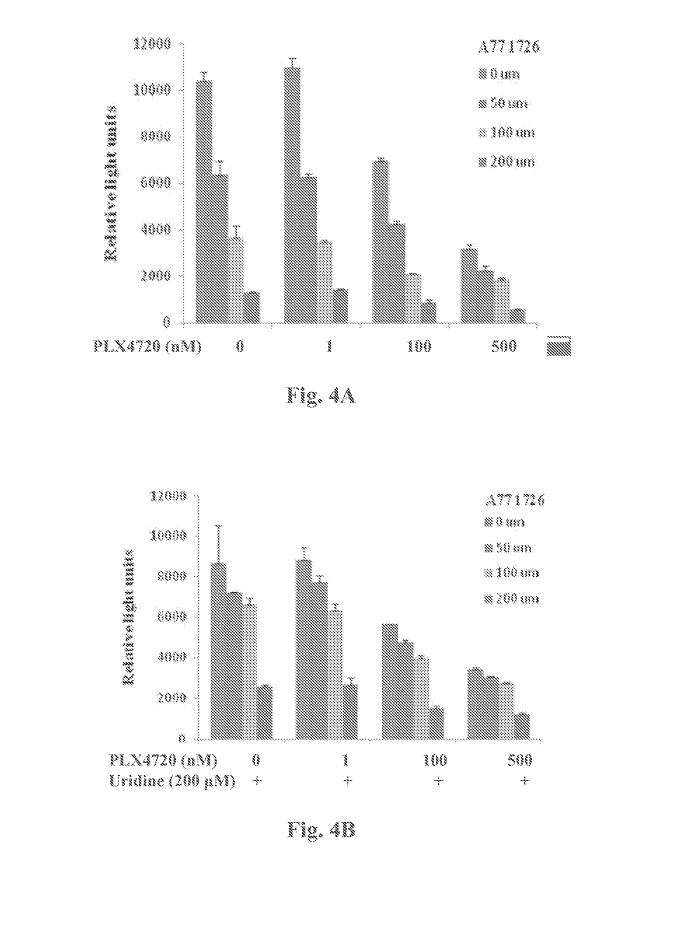

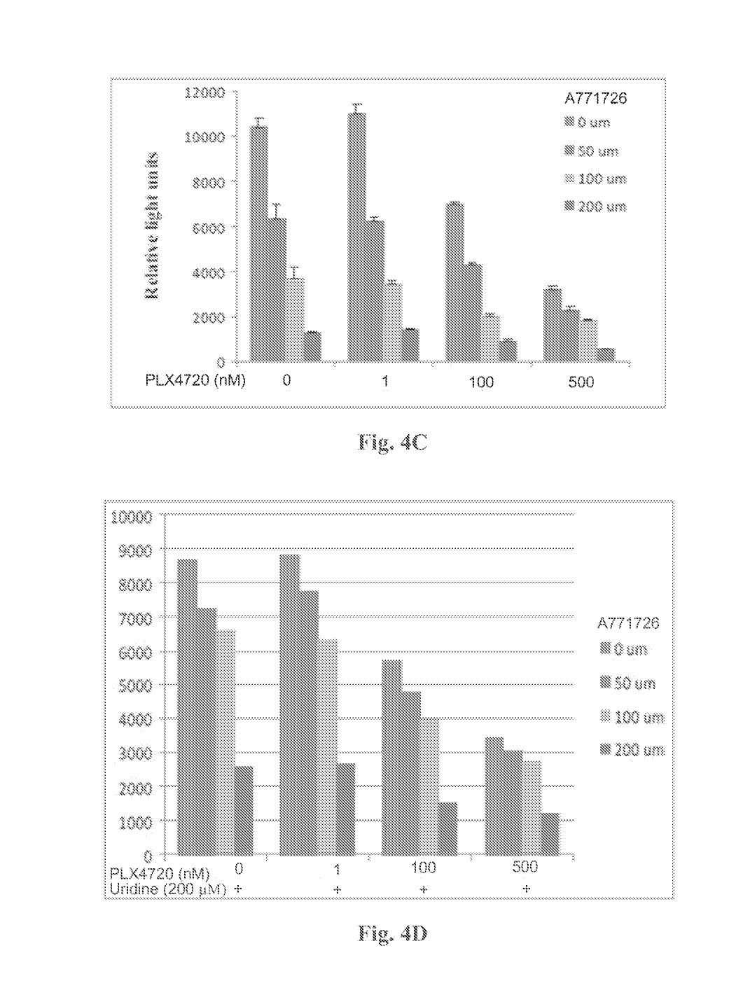

We next tested whether inhibition of S6K1 activity contributed to the anti-proliferative activity of A77 1726 on A375 cells. As shown in FIGS. 4A and 4C, A77 1726 inhibited the proliferation of A375 in a dose-dependent manner, with an IC₅₀ value of approximately 65 μM. Exogenous uridine only partially blocked the inhibitory effect of A77 1726 used at 50 or 100 μM and had minimal effect in reversing the inhibitory effect of A77 1726 used at 200 LM (FIGS. 4B and 4D). PLX4720 at 1 nM by itself had no effect on A375 cell proliferation and did not affect A77 1726-mediated anti-proliferative effect (FIGS. 4A and 4C). PLX4720 (100 or 500 nM) by itself inhibited A375 proliferation (FIGS. 4A and 4C). When used in combination with A77 1726 in the absence or presence of uridine, PLX4720 had an additive effect on inhibiting the proliferation of A375 cells (FIGS. 4B and 4D).

### Example 4

### Effect of A77 1726 on Cell Cycle Arrest

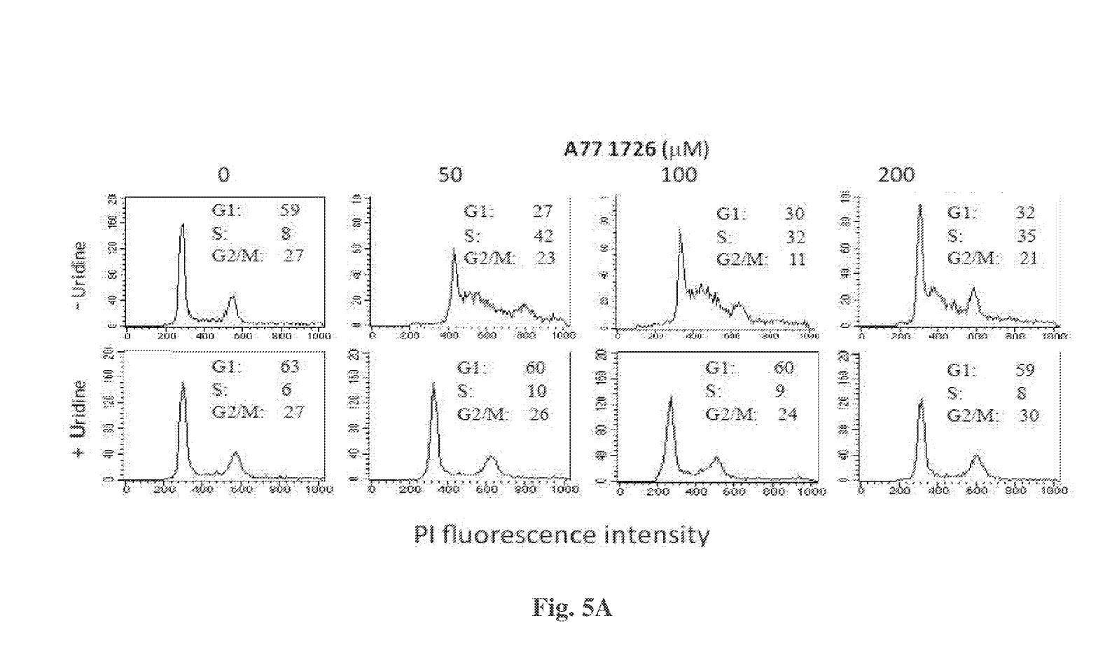

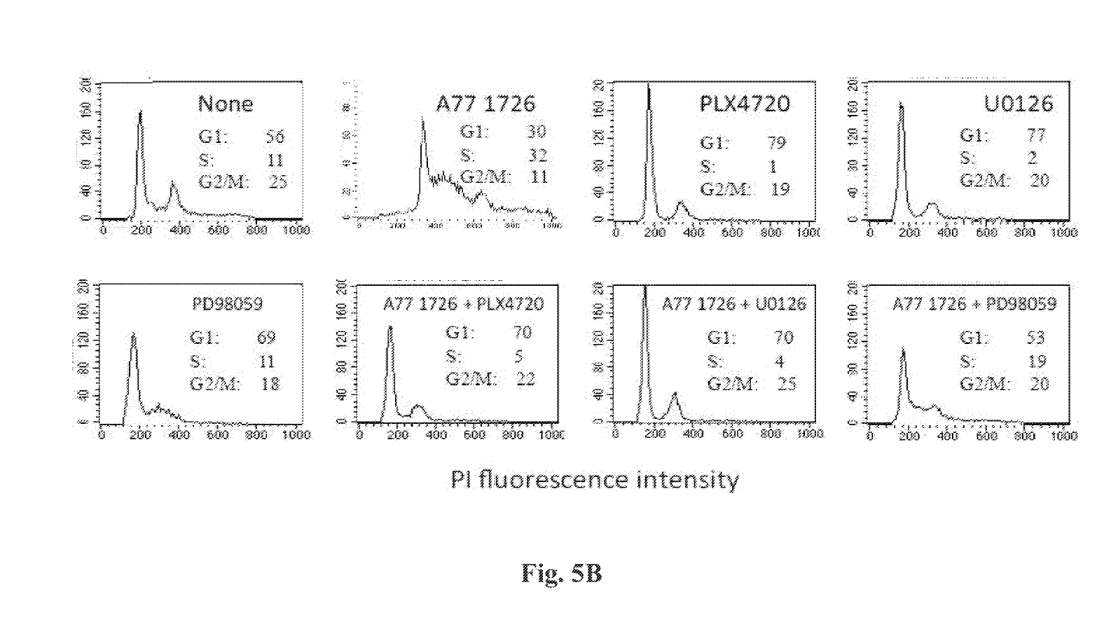

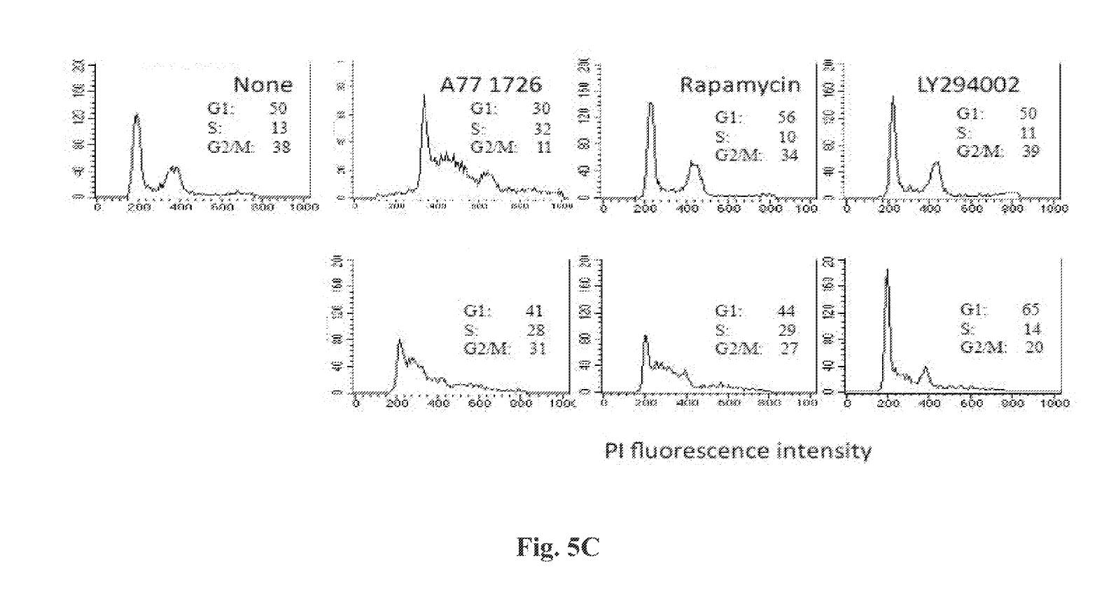

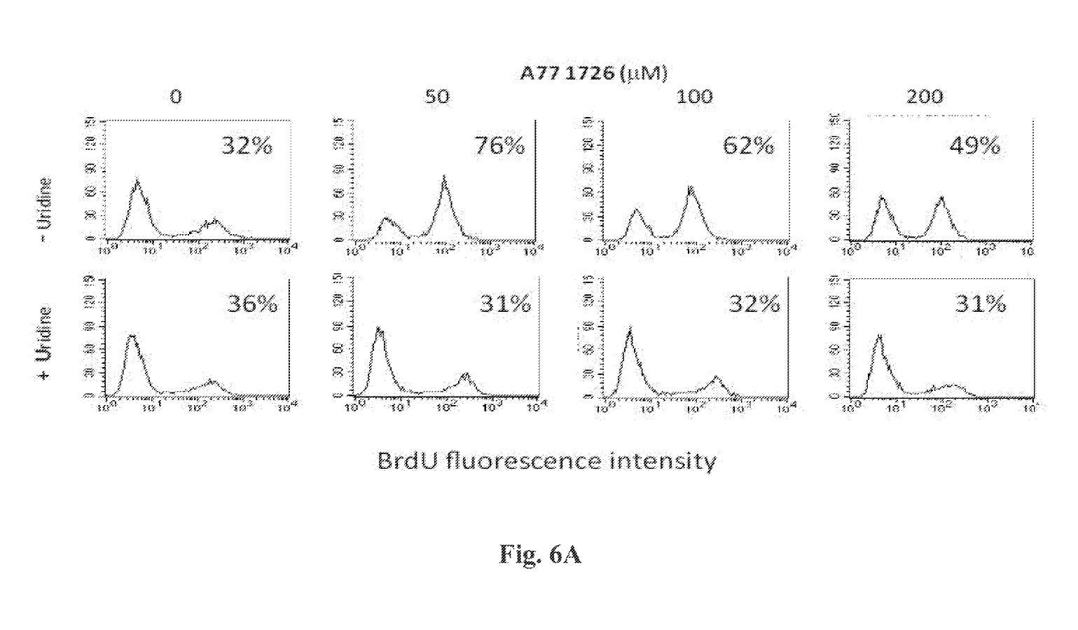

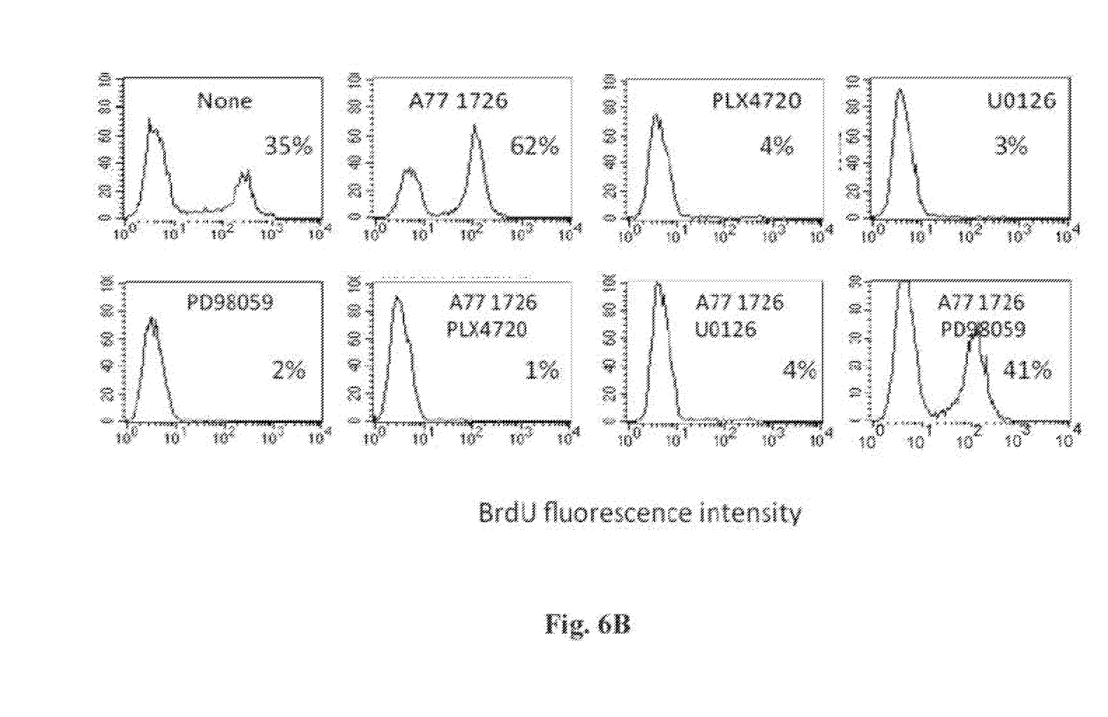

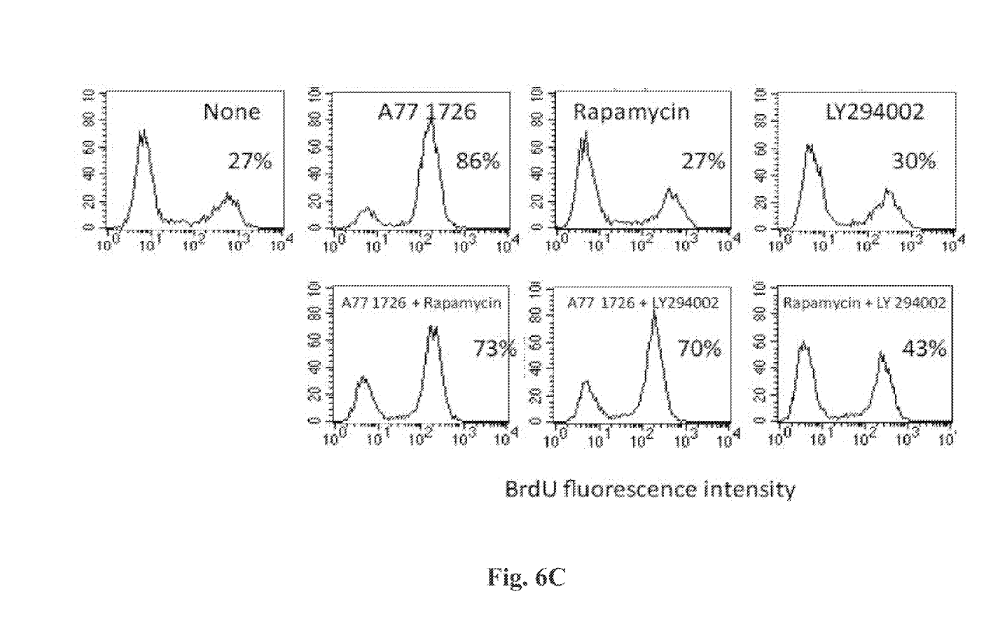

We next tested whether the anti-proliferative effect of A77 1726 was mediated by arresting cell cycle progress. A375 cells grown in 6-well plates were treated with the indicated concentration of A77 1726 and/or uridine for 24 hr and analyzed for cell cycle by PI staining. As shown in FIG. 5A, A77 1726 at 50 μM was sufficient to arrest cell cycle in the S phase. Consistent with this observation, a significantly higher number of cells treated with 50 μM were labeled with BrdU than those treated with A77 1726 at 100 or 200 μM. (FIG. 6A). Uridine alone had little effect in cell cycle arrest, compared to the control cells. Uridine was able to almost normalize cell cycle in A375 cells treated with A77 1726 at three different concentrations. BrdU labeling revealed that uridine was able to block increased BrdU incorporation induced by A77 1726 at 50 and 100 μM but slightly decreased BrdU incorporation in A375 cells treated with A77 1726 at 200 μM each (FIG. 6A).

The inhibitors of the MAP kinase pathways were used to determine whether A77 1726-induced DNA synthesis was mediated by MAP kinase activation. As shown in FIG. 5B, PIA 4720 (1 μM), U0126 (10 μM), PD98059 (10 μM) led to the arrest of the cell cycle in G1 phase in untreated or A77 1726-treated A375 cells. BrdU labeling revealed that PIA 4720 (1 μM) and U0126 (10 μM) alone were able to completely block DNA replication in A375 cells (FIG. 5B), PLX4720 or U0126 was also able to block A77 1726-induced DNA incorporation. Interestingly, PD98059 (10 μM), which only partially blocked A77 1276-induced cell cycle arrest in G1 phase, also partially blocked A77 1726-induced BrdU incorporation. These results suggest that A77 1726 treatment led to accelerated DNA synthesis through MAP kinase activation. Rapamycin or LY249002 alone led to the arrest of cell cycle in the G-1 phase (FIG. 5C). However, rapamycin and LY294002 were unable to promote cell cycle progress of A77 1726-treated A375 cells. BrdU labeling revealed that LY294002 or rapamycin alone did not significantly affect DNA synthesis. Both inhibitors slightly attenuated A77 1726-stimulated DNA synthesis (FIG. 6C).

### Example 5

### Effect of A77 1726 on CAD Phosphorylation

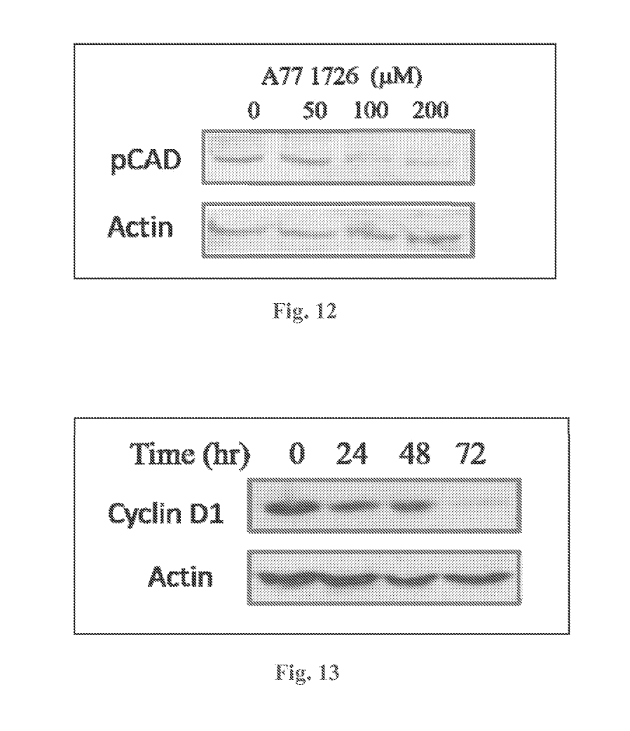

Two recent studies have demonstrated that S6K1 phosphorylates carbomoyl-phosphate synthetase 2 (CAD), a rate-limiting enzyme involved in pyrimidine nucleotide synthesis and stimulates its enzymatic activities. The studies have demonstrated the leflunomide and its active metabolite A 77 1726 target S6K1. The ability of 177 1726 to inhibit phosphorylation of CAD at the Ser-1859 site was tested. A375 melanoma tumor cell line cultures were pretreated with the 0, 50, 100 or 200 μM A 771726 for 2 hours. Total cell lysates were prepared and analyzed for the phosphorylation of CAD by a phosphor-specific antibody. As shown in FIG. 12, A 77 1726 inhibited phosphorylation in a dose-dependent manner. These results suggest that in addition to the ability to inhibit DHO-DHase, lefunomide and A77 1726 may inhibit pyrimidine nucotide synthesis by inhibiting S6K1-mediated CAD phosphorylation and enzymatic activity.

### Example 6

### Effect of A77 1726 on Cyclin D1 Expression

S6K 1 has been implicated in playing an important role in regulating expression of cyclin D1. Cyclin D1 expression was tested in the presence of A 77 1726. A375 cells were treated with 200 mM A 77 1726 for 0, 24, 48 and 72 hours. As shown in FIG. 13, cyclin D1 expression was inhibited by A 77 1726 in a time-dependent manner. The results collectively suggest that S6K1 is a molecular target of A 77 1726, and that inhibition of S6K2 by A77 1726 plays an important role in mediating anti-proliferative activity.

### Example 7

### Summary of Examples 2-6

Mechanisms by which A77 1726, the active metabolite of a novel immunosuppressive drug, lefluomide, exerts its immunosuppression and anti-proliferative activity, are incompletely understood. Here we show that A77 1726 rapidly inhibited the phosphorylation of ribosomal protein S6 but induced the phosphorylation of AKT, p70 S6 (S6K1), ERK1/2, and MEK in an A375 melanoma cell line in a dose- and time-dependent manner. In vitro kinase assay revealed that leflunmide and A77 1726 inhibited S6K1 activity, with the IC₅₀ values of approximately 55 μM and 80 μM, respectively. LY294002 (a PI-3 kinase inhibitor) was able to block leflunomide- and A77 1726-induced AKT activation but not MAP kinase phosphorylation, whereas PPP (an inhibitor of IGF-1 receptor tyrosine kinase) was able to partially block AKT, S6K1, ERK1/2, and MEK phosphborylation in A77 1726-treated cells. B-Raf and MEK kinase inhibitors were able to block A77 1726-induced ERK1/2 and MEK but not AKT phosphorylation. The ability of A77 1726 to inhibit A375 cell proliferation was partially blocked by addition of exogenous uridine into cell culture. PLX4720, an inhibitor of mutant B-Raf kinase, inhibited A375 cell proliferation, and combining with A77 1276, even in the presence of exogenous uridine, achieved an additive anti-proliferative effect on A375 cells. Arrest of the cell cycle in the S phase in A375 cells could be blocked by addition of exogenous uridine and by the MAP kinase pathway inhibitors but not by LY294002. Taken together, our studies have identified S6K1 as a novel target of A77 1726 and demonstrated that inhibition of S6K1 leads to the feedback activation of the MAP kinase pathway and the upstream segment of the PI-3 kinase pathway. A77 1726 exerts its anti-proliferative effect in part by inhibiting S6K1 kinase activity.

### Example 8

### Discussion of Examples 2-6

S6K1 is a member of a group of serine/threonine protein kinases A, G, and C (AGC) family, including AKT and mTOR. S6K1 is one of the predominant effectors of the mTORC1 (mammalian target of rapamycin complex 1) (20). The mTORC1-56K1 pathway plays an important role in regulating protein synthesis, cell growth, metabolism, and ageing (20). S6K1 is overexpressed or activated in primary liver neoplasms, ovarian cancers, and many other types of malignancy due to the gene mutations in the PI-3 kinase pathway (20; 21) S6K1 gene amplification occurs in 10% of breast cancers and is associated with a poor prognosis (22). S6K1 serves as a biomarker to predict breast cancer response to rapamycin (23). There have been considerable efforts in search for the specific inhibitors to target this important player in the mTORC1-S6K1 pathway. Numerous small molecule compounds that inhibit S6K1 alone or both S6K1 and AKT are at the early stage of clinical trials for anticancer therapy (20). Our present study provides unambiguous evidence that A77 1726 and leflunomide were able to inhibit S6K1 activity, subsequently leading to the inhibition of S6 phosphorylation as well as the feedback activation of AKT and MAP kinase pathways through the IGF-1 receptor.

The IC₅₀ values of leflunomide and A77 1726 to inhibit S6 phosphorylation in cell culture was approximately between 50-75 μM, consistent with the results obtained from in vitro kinase assay revealing the IC₅₀ value of leflunomide and A77 1726 to inhibit S6K1 approximately at 55 and 80 μM, respectively. A77 1726 or leflunomide may also inhibit S6K2 but is unlikely able to inhibit other AGC kinases such as AKT and mTOR. The IC₅₀ values of leflunomide and A77 1726 required to inhibit S6K1 are physiologically relevant. The phamacokinetics of lelfunomide favorably fit its potential use in oncology. Plasma concentrations of A77 1726 in rheumatoid arthritis patients treated with leflunomide (20 mg/day) can reach 200 μM (24). The serum concentrations of A77 1726 in mice treated with leflunomide at a dose of 35 mg, had a remarkably long half-life of 15 hr. A77 1726 peaks at 500 μM within 4 hr and remains at 250 μM at 24 hr after a single dose of 35 mg/kg of leflunomide (25). Both these concentrations are sufficient to inhibit S6K1 activity. The IC₅₀ values of A77 1276 required to inhibit S6K1 are equivalent to its ability to inhibit PDGF receptor and Src family tyrosine kinases (5). Our previous studies have shown that leflunomide treatment was able to inhibit tyrosine phosphorylation in an lpr/lpr mouse model (4) and in an C6 rat glioma tumor xenograft model (5). We anticipate that the dose of leflunomide used under those schemes will allow the concentration of A77 1726 in tumor tissue to exceed its ICs values.

Several prior studies have demonstrated that A77 1726 is capable of inhibiting the activation of the PI3K pathway. For example, Baumann et al (15) reported that the phosphorylation of AKT^{T308}, AKT^{S473}, 4E-BP^{T37/46}, and S6K1^{T389} is inhibited in H929 and OPM-2 myeloma cell lines after incubation for 24 and 48 hr with A77 1726 (200 μM). Since A77 1726 is unable to inhibit the phosphorylation of these proteins at 4 hr after drug addition to cell culture (15), the inhibitor), effect of A77 1726 could result from an indirect effect. Liacini et al. (16) reported that A77 1726 is able to weakly inhibit PDK1 and AKT in a renal CCD1105 cell line and primary human tubular cells. These investigators proposed that A77 1726 may target PDK1 or AKT. Sawamukai et al. (14) reported that A77 1726 at the concentrations of 100 and 200 μM is able to inhibit the c-Kit ligand-induced PDK^{S241}, AKT^{T308} GSK3β^{S9} in human mast cells. It is not clear whether inhibition of PDK1 and AKT phosphorylation by A77 1726 in human mast cells resulted from the inhibition of the c-Kit growth factor receptor tyrosine kinase. In contrast, our present study demonstrated that leflunomide and A77 1726 were able to inhibit S6K1 activity in three cell lines (A375, BT-20, and MCF-7). This led to AKT activation through a negative feedback mechanism. In support of this notion, inhibition of PI-3 kinase activity by LY294002 and inhibition of IGF-1 receptor by PPP were able to block A77 1726-induced AKT phosphorylation (FIG. 3B). Leger et al. (26) reported that leflunomide used at 10 μM is able to induce the phosphorylation of AKT^{T308} in two erythroleukemia cell lines (HEL and K562), which could be blocked by LY294002. It is unclear whether increased AKT^{T308} leflunomide-treated in erytheroleukemia cells also involves a feedback activation mechanism. Nevertheless, our study did not find that leflunomide and A77 1726 were able to increase AKT^{S308} phosphrylation in A375 and MCF-7 cells (data not shown).

While both rapamycin and A77 1276 were able to induce the feedback activation of the PI-3 kinase pathway, rapamycin and A77 1726 had differential effect in activating the MAP kinase pathway in A375 cells. ERK1/2 and MEK phosphorylation was rapidly and potently induced in A375 cells by A77 1726 and leflunomide but only minimally induced by rapamycin (FIG. 2). It is not clear whether this is because A77 1726 and rapamycin target different molecules in the MTORC1-S6K1 pathway. Unexpectedly, we found that there was no increased Raf-1 phosphorylation in A375 cells treated with A77 1726 or leflunomide. In contrast, a prior study showed that A77 1726 (50 and 100 μM) is able to induce Raf-1 phosphorylation in a BON human gastrointestinal carcinoid cell line (27) The reason for this discrepancy is not clear. Regardless, it appears that A77 1726 is not a B-Raf kinase inhibitor since A77 1726 was unable to induce Raf-1 transactivation and ERK phosphorylation in BRAF wild-type cell lines (Xu, X., unpublished observation).

Previous studies have shown that A77 1726 is able to arrest cell cycle progress either at G1 or S phase, depending on cell types. For example, Ruckemann et al. reported that A77 1726 arrests cell cycle progress in the G1 phase in lymphocytes (8). Baumann et al. reported that A77 1726 is able to arrest cell cycle in the G1 phase in several myeloma cell lines (15). In contrast, several other studies have demonstrated that A77 1727 arrests cell cycle progress in the S phase of cancer cells: Cook et al. reported that A77 1726 was able to arrest the cell cycle in the S phase in a human gastrointestinal carcinoid cell line; Hail et al. (28) reported that A77 1726 dramatically arrested the cell cycle in the S phase in human prostate cancer cell line and cutaneous squamous cancer cell lines. Huang et al. (29) suggested that cell cycle arrest in the S phase in K562 cells relies on mutant p53. Our present study demonstrated that A77 1726 led to cell cycle arrest in the S phase in wild-type p53 A375 cells. Our mechanistic study suggests that A77 1726 may induce a rapid entry of the cell cycle into the S phase in A375 cells through the MAP kinase activation. Indeed, MAP kinase pathway inhibitors (U0126, PLX2720, and Bay 43-9006) were able to arrest the cell cycle in the G1 phase in untreated and A77 1726-treated A375 cells. Interestingly, we found that exogenous uridine was able to largely relieve the arrest of the cell cycle in S phase but was unable to completely normalize cell cycle progress, compared to untreated control. We speculate that depletion of pyrimidine nucleotide pools in A77 1726-treated cells prevents the completion of DNA synthesis and chromosomal duplication.

The anti-proliferative activity of A77 1726 has been well documented. However, the underlying molecular mechanisms are not fully understood. Depletion of pyrimidine nucleotide pools in vitro in cell culture by A77 1726, particular at low concentrations (<50 μM), is largely responsible for its anti-proliferative activity. Uridine was able to block 80% of the inhibitory effect of A77 1726 used at 50 or 100 μM but only blocked about 20% inhibitory effect with A77 1726 at 200 μM. These results suggest that A77 1726 used at high concentrations largely exerts its anti-proliferative activity independent of its anti-pyrimidine mechanism. In the present study, we demonstrated that A77 1726 was able to inhibit S6K1 activity. Since its downstream effector, ribosomal protein S6, plays a critical role in protein synthesis, A77 1726 may exert its antiproliferative activity in part by inhibiting 56 kinase activation. Though our previous studies demonstrated that A77 1726 is able to inhibit protein tyrosine kinases (PTK), it is unlikely that inhibition of PTK is responsible for its antiproliferative activity since the MAP kinase pathway in A375 cells was not inhibited but rather activated by A77 1726. Activation of the MAP kianse pathway may actually antagonize its anti-proliferative effect mediated through the inhibition of S6K1. In support of this notion, we found that A77 1726 in combination with PLX4720 was able to achieve an additive effect on inhibiting cell proliferation. These observations are consistent with a prior study. (17) showing that PLX4720 at 1 nM by itself does not inhibit A375 cell proliferation, but at 100 and 500 nM cooperates with A77 1726 to inhibit the proliferation of A375 and Hs294T cells.

In summary, our investigation on the mechanisms of action of letiunomide has led to the identification of S6K1 as a novel molecular target of this immunomodulatory drug. Because of the importance of S6K1 in tumor cell proliferation, leflunomide may have potential to be developed as a novel anticancer drug for treating certain types of cancer in which the mTOR-S6K1 pathway is hyperactivated.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

### 4. REFERENCES

1. Zhang S, Yu D: PI(3)king apart PTEN's role in cancer. Clin Cancer Res 16:4325-4330
2. Wander S A, Hennessy B T, Slingerland J M: Next-generation mTOR, inhibitors in clinical oncology: how pathway complexity informs therapeutic strategy. J Clin Invest 121:1231-1241
3. Emerling B M, Akcakanat A: Targeting PI3K/mTOR signaling in cancer. Cancer Res 71:7351-7359
4. Xu X, Blinder I., Shen J, Gong H, Finnegan A, Williams J W, Chong A S: In vivo mechanism by which leflunomide controls lymphoproliferative and autoimmune disease in MRL/MpJ-lpr/lpr mice. J. Immunol 159:167-174, 1997
5. Xu X, Shen J, Mall J W, Myers J A, Huang W, Blinder L, Saclarides T J, Williams J W, Chong A S: In vitro and in vivo antitumor activity of a novel immunomodulatory drug, leflunomide: mechanisms of action. Biochem Pharmacol 58:1405-4413, 1999
6. Xu X, Williams J W, Gong H, Finnegan A, Chong A S: Two activities of the immunosuppressive metabolite of leflunomide, A77 1726. Inhibition of pyrimidine nucleotide synthesis and protein tyrosine phosphorylation. Biochem Pharmacol 52:527-534, 1996
7. Xu X, Williams J W, Bremer E G, Finnegan A, Chong A S: Inhibition of protein tyrosine phosphorylation in T cells by a novel immunosuppressive agent, leflunomide. J Biol Chem 270:12398-42403, 1995
8. Ruckemann K, Fairbanks L D, Carrey E A, Hawrylowicz C M, Richards D F, Kirschbaum B, Simmonds H A: Leflunomide inhibits pyrimidine de novo synthesis in mitogen-stimulated T-lymphocytes from healthy humans. J Biol Chem 273:21682-21691, 1998
9. Elder R T, Xu X, Williams J W, Gong H, Finnegan A, Chong A S: The immunosuppressive metabolite of leflunomide, A77 1726, affects murine T cells through two biochemical mechanisms. J Immunoi 159:22-27, 1997
10. Siemasko K, Chong A S, Jack H M, Gong H, Williams J W, Finnegan A: Inhibition of JAK3 and STAT6 tyrosine phosphorylation by the immunosuppressive drug leflunomide leads to a block in IgG1 production. J Immunol 160:1581-1588, 1998
11. Siemasko K F, Chong A S, Williams J W, Bremer E G, Finnegan A: Regulation of B cell function by the immunosuppressive agent leflunomide. Transplantation 61:635-642, 1996
12. Williamson R A, Yea C M, Robson P A, Curnock A P, Gadher S. Hambleton A B, Woodward K, Bruneau J M, Hambleton P. Spinella-Jaegle S, Morand P, Courtin O, Sautes C, Westwood R, Hercend T, Kuo E A, Ruuth E: Dihydroorotate dehydrogenase is a target for the biological effects of leflunomide. Transplant Proc 28:3088-3091, 1996
13. Bruneau J M, Yea C M, SpineIla-Jaegle S. Fudali C, Woodward K, Robson P A, Sautes C, Westwood R, Kuo E A, Williamson R A, Ruuth E: Purification of human dihydro-orotate dehydrogenase and its inhibition by A77 1726, the active metabolite of leflunomide. Biochem J 336 (Pt 2):299-303, 1998
14. Sawamukai N, Saito K, Yarnaoka K, Nakayamada S, Ra C, Tanaka Y: Lefiunomide inhibits PDK1/Akt pathway and induces apoptosis of human mast cells. J Immunol 179:6479-6484, 2007
15. Baumann P. Mandl-Weber 5, Volkl A, Adam C, Bumeder I, Oduncu. F. Schmidmaier R: Dihydroorotate dehydrogenase inhibitor A771726 (leflunomide) induces apoptosis and diminishes proliferation of multiple myeloma cells. Mol Cancer Ther 8:366-375, 2009
16. Liacini A, Seamone M E, Muruve D A, Tibbles L A: Anti-BK virus mechanisms of sirolimus and leflunomide alone and in combination: toward a new therapy for BK virus infection. Transplantation 90:1450-1457
17. White R M, Cech J, Ratanasirintrawoot S, Lin C Y, Rahl P B, Burke C J, Langdon E, Tomlinson M L, Mosher J, Kaufman C, Chen F, Long H K, Kramer M, Datta S, Neuherg D, Granter S, Young R A, Morrison S, Wheeler G N, Zon L I: DHODH modulates transcriptional elongation in the neural crest and melanoma. Nature 471:518-522
18. Shawver L K, Schwartz D P, Mann E, Chen H, Tsai J, Chu L, Taylorsont Longhi M, Meredith S, Germain L, Jacobs J S, Tang C, Ullrich A, Berens M E, Hersh E, McMahon G, Hirth K P, Powell T J: Inhibition of platelet-derived growth factor-mediated signal transduction and tumor growth by N-[4-(trifluoromethyl)-phenyl]5-methylisoxazole-4-carboxamide. Clin Cancer Res 3:1167-1177, 1997
19. Carracedo A, Ma L, Teruya-Feldstein J, Rojo F, Salmena L, Alimonti A. Egia A, Sasaki A T, Thomas G, Kozma S C, Papa A, Nardella C, Cantley L C, Baselga Pandolfi P P: Inhibition of mTORC1 leads to MAPK pathway activation through a PI3K-dependent feedback loop in human cancer. J Clin Invest 118:3065-3074, 2008
20. Fenton T R, Gout I T: Functions and regulation of the 70 kDa ribosomal S6 kinases. Int J Biochem Cell Biol 43:47-59
21. Sahin F, Kannangai R, Adegbola O, Wang i, Su G, Torbenson M: mTOR and P70 S6 kinase expression in primary liver neoplasms. Clin Cancer Res 10:8421-8425, 2004
22. Perez-Tenorio G, Karlsson E, Waltersson M A, Olsson B, Holmlund B, Nordenskjold B, Formander T, Skoog L, Stal O: Clinical potential of the rriTOR targets S6K1 and S6K2 in breast cancer. Breast Cancer Res Treat 128:713-723
23. Noh W C, Mondesire W H, Peng J, Jian W, Zhang H, Dong J, Mills G B, Hung M C, Meric-Bernstam F: Determinants of rapamycin sensitivity in breast cancer cells. Clin Cancer Res 10:1013-1023, 2004
24. Chan V, Charles B G, Tett S E: Population pharmacokinetics and association between A77 1726 plasma concentrations and disease activity measures following administration of leflunomide to people with rheumatoid arthritis. Br J Clin Pharmacol 60:257-264, 2005
25. Chong A S, Huang W, Liu W, Luo J. Shen J, Xu W, Ma L, Blinder L, Xiao F, Xu X, Clardy C, Foster P, Williams J A: In vivo activity of leflunomide: pharmacokinetic analyses and mechanism of immunosuppression. Transplantation 68:100-109, 1999
26. Leger D Y, Liagre B, Beneytout J L: Low dose leflunomide activates PI3K/Akt signalling in erythroleukemia cells and reduces apoptosis induced by anticancer agents. Apoptosis 11:1747-1760, 2006
27. Cook M R, Pinchot S N, Jaskula-Sztul R, Luo J, Kunnimalaiyaan M, Chen H: Identification of a novel Raf-1 pathway activator that inhibits gastrointestinal carcinoid cell growth. Mol Cancer her 9:429-437
28. Hail N, Jr., Chen P, Bushman L R: Teriflunomide (leflunomide) promotes cytostatic, antioxidant, and apoptotic effects in transformed prostate epithelial cells: evidence supporting a role for terifiunomide in prostate cancer chemoprevention. Neoplasia 12:464-475
29. Huang M, Wang Y, Collins M, Mitchell B S, Graves L M: A77 1726 induces differentiation of human myeloid leukemia K562 cells by depletion of intracellular CTP pools. Mol Pharmacol 62:463-472, 2002
30. O'Donnell, E. F., Kopparapu, P. R., Koch, D. C., Jang, H. S., Phillips, J. L., Tanguay, R. L., Kerkvliet, N. I, and Kolluri, S. K. PLoS One 7[7], e40926.

1. A method for treating an S6K-overexpressing cancer, comprising administering a therapeutically effective amount of leflunomide to a subject in need thereof.

2. The method of claim 1, wherein administering the therapeutically effective amount of leflunomide inhibits or reduces cell proliferation in the S6K-overexpressing cancer.

3. The method of claim 1, wherein administering the therapeutically effective amount of leflunomide inhibits or reduces activity of an S6K protein.

4. The method of claim 3, wherein the S6K protein is S6K1.

5. The method of claim 1, wherein administering the therapeutically effective amount of leflunomide inhibits or reduces phosphorylation of a ribosomal S6 protein.

6. The method of claim 1, wherein administering the therapeutically effective amount of leflunomide induces phosphorylation of a protein selected from the group consisting of: AKT, S6K1, ERK1/2, and MEK.

7. The method of claim 1, wherein the S6K-overexpressing cancer is a breast cancer.

8. The method of claim 1, wherein the S6K-overexpressing cancer has an amplification of an S6K, gene or a hyperactivated mTOR-S6K1 pathway.

9. The method of claim 1, further comprising administering a therapeutically effective amount of an agent, wherein the agent in combination with leflunomide reduces the proliferation of cancer cells in the S6K-overexpressing cancer more than administering leflunomide alone.

10. The method of claim 9, wherein the agent is PLX4720.
